(19) United States
(12) Patent Application Publication
(10) Pub. No.: US 2026/0325152 A1
(43) Pub. Date: Oct. 22, 2026

(54) LITHOTRIPSY CATHETER WITH SHARED EMITTER BETWEEN MULTIPLE CHANNELS

(71) Applicant: Shockwave Medical, Inc., Santa Clara, CA (US)

(72) Inventors: Keian Rennel Serrano Castro, San Jose, CA (US); Carlos H. Lima, Campbell, CA (US); Dexter Alexander Lew De Castro, Santa Clara, CA (US)

(73) Assignee: Shockwave Medical, Inc., Santa Clara, CA (US)

(21) Appl. No.: 19/645,067

(22) Filed: Apr. 10, 2026

Related U.S. Application Data
(60) Provisional application No. 63/790,518, filed on Apr. 17, 2025.

(51) Int. Cl. A61B 17/22

(52) U.S. Cl. CPC A61B 17/22022; A61B 2017/22008; A61B 2017/22018; A61B 2017/22025; A61B 2017/22069

(57) ABSTRACT

This disclosure provides methods, devices, and systems for energy-based lithotripsy. The present implementations more specifically relate to lithotripsy catheters with shared emitters between multiple channels. In some aspects, one or more emitters on the same catheter may be coupled to multiple channels. For example, a catheter may include three emitters coupled to two channels such that the first emitter is coupled to the first channel, the second emitter is coupled to the second channel, and the third emitter is coupled to both the first and second channels. In this way, the third emitter can generate acoustic waves in response to each energy pulse output by an energy source (on any of the channels). By contrast, the first emitter can generate acoustic waves only in response to energy pulses on the first channel and the second emitter can generate acoustic waves only in response to energy pulses on the second channel.

### CROSS-REFERENCE TO RELATED APPLICATIONS

This patent application claims priority and benefit under 35 U.S.C. § 119(e) to U.S. Provisional Patent Application No. 63/790,518, filed Apr. 17, 2025, and entitled “INTRAVASCULAR LITHOTRIPSY (IVL) CATHETER WITH SHARED EMITTER BETWEEN MULTIPLE CHANNELS.” The disclosure of the prior Application is considered part of and is incorporated by reference in this Patent Application.

### TECHNICAL FIELD

This disclosure relates generally to medical systems, and specifically to lithotripsy catheters with shared emitters between multiple channels.

### DESCRIPTION OF RELATED ART

The accumulation of calcium in a patient’s blood vessels, tissues, or other organs can cause calcification that may disrupt organ function and lead to health issues for the patient. Various treatment options have been developed to address calcification and restore organ function. One such treatment is lithotripsy. In particular, energy-based lithotripsy (such as intravascular lithotripsy (IVL)) uses acoustic pressure to break up the calcified regions. For example, a device such as a catheter is advanced within the patient’s body to a position adjacent to the treatment area. Such device is configured to generate acoustic waves, such as ultrasonic short pulse waves (also known as “shock waves”), which propagate outward from the device to modify the calcified regions. The acoustic pressure can crack and disrupt the calcified regions without harming the surrounding blood vessels, tissues, or other organs.

As used herein, the term “emitter” refers any device or component of a medical instrument that can transfer energy (such as electrical or optical energy) into a medium resulting in the generation of acoustic waves. An emitter can be formed at a particular location (e.g., longitudinally) along a length (or axis) of a catheter. For electrohydraulic generation of acoustic waves, a single emitter may include one or more pairs of electrodes positioned radially around the axis of the catheter. A lithotripsy catheter can include one or more emitters positioned along its axis. Some lithotripsy catheters have multiple emitters that can be used to treat longer lesions and/or multiple lesions in a shorter amount of time (such as by reducing the amount of moving or repositioning of the catheter). However, the strength and/or efficacy of the acoustic waves generally depends on the amount of energy (or voltage) that can be supplied to each pair of electrodes, which may be limited by existing power generation technologies. Thus, there is a need to increase the sonic output of a lithotripsy catheter without increasing its energy budget.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter of this disclosure can be implemented in a medical instrument. The medical instrument includes an elongate shaft having a plurality of transmission media disposed thereon forming a first channel and a second channel; a first emitter disposed on the elongate shaft and configured to produce acoustic waves responsive to energy pulses on the first channel; a second emitter disposed on the elongate shaft and configured to produce acoustic waves responsive to energy pulses on the second channel; and a third emitter disposed on the elongate shaft and configured to produce acoustic waves responsive to the energy pulses on each of the first channel and the second channel.

Another innovative aspect of the subject matter of this disclosure can be implemented in a method performed by a pulse generator for a medical system. The method includes steps of outputting one or more first energy pulses on a first channel coupled to a first emitter and a second emitter disposed on an elongate shaft, where each of the first and second emitters is configured to produce acoustic waves responsive to the one or more first energy pulses; and outputting one or more second energy pulses on a second channel coupled to the second emitter and a third emitter disposed on the elongate shaft, where each of the second and third emitters is configured to produce acoustic waves responsive to the one or more second energy pulses.

### BRIEF DESCRIPTION OF THE DRAWINGS

The present implementations are illustrated by way of example and are not intended to be limited by the figures of the accompanying drawings.

FIG. 1 shows an example medical system, according to some implementations.

FIG. 2 shows a block diagram of an example catheter system, according to some implementations.

FIG. 3 shows a circuit diagram of an example catheter system, according to some implementations.

FIG. 4 shows another block diagram of an example catheter system, according to some implementations.

FIG. 5A shows another circuit diagram of an example catheter system, according to some implementations.

FIG. 5B shows another circuit diagram of an example catheter system, according to some implementations.

FIG. 6 shows an example emitter assembly, according to some implementations.

FIGS. 7A and 7B show perspective views of an example emitter disposed around an elongate shaft, according to some implementations.

FIG. 8 shows a perspective view of an example conductive band for an emitter, according to some implementations.

FIG. 9A shows another perspective view of an example emitter disposed around an elongate shaft, according to some implementations.

FIG. 9B shows a sectional view of the emitter depicted in FIG. 9A.

FIGS. 10A–10C show an example catheter assembly, according to some implementations.

FIG. 11 shows an example wiring diagram for the catheter assembly depicted in FIGS. 10A–10C, according to some implementations.

FIGS. 12A and 12B show another example catheter assembly, according to some implementations.

FIG. 13 shows an example wiring diagram for the catheter assembly depicted in FIGS. 12A and 12B, according to some implementations.

FIGS. 14A–14C show example emitter configurations, according to some implementations.

FIGS. 15A and 15B show another example catheter assembly, according to some implementations.

FIG. 16 shows an example wiring diagram for the catheter assembly depicted in FIGS. 15A and 15B, according to some implementations.

FIGS. 17A and 17B show another example catheter assembly, according to some implementations.

FIG. 18 shows a block diagram of an example pulse generator for a medical system, according to some implementations.

FIG. 19 shows an illustrative flowchart depicting an example operation that can be performed by a pulse generator for a medical system, according to some implementations.

### DETAILED DESCRIPTION

In the following description, numerous specific details are set forth such as examples of specific components, circuits, and processes to provide a thorough understanding of the present disclosure. The term “coupled” as used herein means connected directly to or connected through one or more intervening components or circuits. The terms “electronic system” and “electronic device” may be used interchangeably to refer to any system capable of electronically processing information. Also, in the following description and for purposes of explanation, specific nomenclature is set forth to provide a thorough understanding of the aspects of the disclosure. However, it will be apparent to one skilled in the art that these specific details may not be required to practice the example implementations. In other instances, well-known circuits and devices are shown in block diagram form to avoid obscuring the present disclosure. Some portions of the detailed descriptions which follow are presented in terms of procedures, logic blocks, processing and other symbolic representations of operations on data bits within a computer memory.

These descriptions and representations are the means used by those skilled in the data processing arts to most effectively convey the substance of their work to others skilled in the art. In the present disclosure, a procedure, logic block, process, or the like, is conceived to be a self-consistent sequence of steps or instructions leading to a desired result. The steps are those requiring physical manipulations of physical quantities. Usually, although not necessarily, these quantities take the form of electrical or magnetic signals capable of being stored, transferred, combined, compared, and otherwise manipulated in a computer system. It should be borne in mind, however, that all of these and similar terms are to be associated with the appropriate physical quantities and are merely convenient labels applied to these quantities.

Unless specifically stated otherwise as apparent from the following discussions, it is appreciated that throughout the present application, discussions utilizing the terms such as “accessing,” “receiving,” “sending,” “using,” “selecting,” “determining,” “normalizing,” “multiplying,” “averaging,” “monitoring,” “comparing,” “applying,” “updating,” “measuring,” “deriving” or the like, refer to the actions and processes of a computer system, or similar electronic computing device, that manipulates and transforms data represented as physical (electronic) quantities within the computer system’s registers and memories into other data similarly represented as physical quantities within the computer system memories or registers or other such information storage, transmission or display devices.

Certain standard anatomical terms of location may be used herein to refer to the anatomy of animals, and namely humans, with respect to the example implementations. Although certain spatially relative terms, such as “outer,” “inner,” “upper,” “lower,” “below,” “above,” “vertical,” “horizontal,” “top,” “bottom,” and similar terms, are used herein to describe a spatial relationship of one element, device, or anatomical structure to another device, element, or anatomical structure, it is understood that these terms are used herein for ease of description to describe the positional relationship between elements and structures, as illustrated in the drawings. It should be understood that spatially relative terms are intended to encompass different orientations of the elements or structures, in use or operation, in addition to the orientations depicted in the drawings. For example, an element or structure described as “above” another element or structure may represent a position that is below or beside such other element or structure with respect to alternate orientations of the subject patient, element, or structure, and vice-versa. As used herein, the term “patient” may generally refer to humans, anatomical models, simulators, cadavers, and other living or non-living objects.

In the figures, a single block may be described as performing a function or functions; however, in actual practice, the function or functions performed by that block may be performed in a single component or across multiple components, or may be performed using hardware, using software, or using a combination of hardware and software. To clearly illustrate this interchangeability of hardware and software, various illustrative components, blocks, modules, circuits, and steps have been described below generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example systems or devices may include components other than those shown, including well-known components such as a processor, memory and the like.

The techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium including instructions that, when executed, performs one or more of the methods described herein. The non-transitory processor-readable data storage medium may form part of a computer program product, which may include packaging materials.

The non-transitory processor-readable storage medium may comprise random access memory (RAM) such as synchronous dynamic random-access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, or executed by a computer or other processor.

The various illustrative logical blocks, modules, circuits and instructions described in connection with the implementations disclosed herein may be executed by one or more processors (or a processing system). The term “processor,” as used herein may refer to any general-purpose processor, special-purpose processor, conventional processor, controller, microcontroller, or state machine capable of executing scripts or instructions of one or more software programs stored in memory.

As used herein, the term “electrode” refers to any electrically conducting element (such as stainless steel, copper, tungsten, platinum, palladium, molybdenum, cobalt, chromium, iridium, an alloy or alloys thereof, such as cobalt-chromium, platinum-chromium, cobalt-chromium-platinum-palladium-iridium, or platinum-iridium, or other metal) that can receive electrical current and pass the current to another electrically conducting element. In the context of the present disclosure, electrodes can be positioned in relation to one another, such as in an arrangement of an inner electrode and an outer electrode. Accordingly, the term “electrode pair” refers to two electrodes positioned in close proximity such that application of a sufficiently high voltage can cause an electrical current to transmit across a gap (also referred to as a “spark gap”) between the electrodes, such as by passing through a conductive fluid or gas. As used herein, the term “emitter” refers to any device or component of a medical instrument that can transfer energy (such as electrical or optical energy) into a medium, resulting in the generation of acoustic waves, such as shock waves and/or ultrasound waves. In the context of the present disclosure, an emitter can refer to one or more electrode pairs formed at a particular location (e.g., longitudinally) along a length (or axis) of a catheter. A spark gap broadly refers to any region of an emitter where current transmits across an electrode pair.

As described above, a lithotripsy catheter can include one or more emitters positioned (e.g., longitudinally) along a length or axis of the catheter. Each emitter can include one or more spark gaps positioned radially around the axis of the catheter. Some lithotripsy catheters have multiple emitters that can be used to treat longer lesions and/or multiple lesions in a shorter amount of time (such as by reducing the amount of moving or repositioning of the catheter). However, the strength and/or efficacy of the acoustic waves generally depends on the amount of energy (or voltage) that can be supplied to each pair of electrodes, which may be limited by existing power generation technologies.

Aspects of the present disclosure recognize that emitters can be added to a catheter, without increasing its energy consumption, by supplying energy to the emitters via different channels. As used herein, the term “channel” refers to any suitable transmission medium, or network thereof, that provides a path for energy (such as electrical or optical energy) to flow between a catheter and an energy source. Thus, the terms “channel” and “conductive channel” may be used herein interchangeably. The term “acoustic wave transducer” refers to any component of a lithotripsy catheter that can convert electrical or optical energy into acoustic waves in a surrounding medium (such as fluid or gas), for example, using electrohydraulic, laser, or piezoelectric generation techniques. Emitters can be coupled to one or more channels so that the number of acoustic wave transducers receiving the same energy pulse does not exceed the capabilities of the energy source. As used herein, For example, up to four electrode pairs can be coupled to a first conductive channel if the energy source can produce at most four shock waves with a single voltage pulse. Another four electrode pairs can be coupled to a second conductive channel so that the electrodes coupled to different channels receive voltage pulses at different times. For example, the energy source may output a series of voltage pulses on the first and second channels in an interleaved fashion, causing the electrode pairs coupled to the first channel to emit shock waves at different times than the electrode pairs coupled to the second channel.

In some aspects, one or more emitters on the same catheter may be coupled to multiple channels (also referred to as “shared emitters”). For example, a catheter may include eight electrode pairs distributed across three emitters such that two of the electrode pairs are formed on a first emitter, another two electrode pairs are formed on a second emitter, and the remaining four electrode pairs are formed on a shared emitter. In some implementations, the two electrode pairs on the first emitter and two of the electrode pairs on the shared emitter may be coupled to a first conductive channel while the two electrode pairs on the second emitter and the other two electrode pairs on the shared emitter are coupled to a second conductive channel. In this way, the shared emitter can generate shock waves (albeit via different pairs of electrodes) in response to each voltage pulse output by the energy source. By contrast, the first emitter can generate shock waves only in response to voltage pulses on the first conductive channel and the second emitter can generate shock waves only in response to voltage pulses on the second conductive channel.

Particular implementations of the subject matter described in this disclosure can be implemented to realize one or more of the following potential advantages. By adding emitters along the length of a catheter, aspects of the present disclosure can increase the acoustic reach or coverage of the catheter. As a result, such catheters can be more effective at treating longer lesions and/or multiple lesions in a shorter amount of time (such as by reducing the amount of moving or repositioning of the catheter). Further, the acoustic waves generated by multiple emitters can constructively interfere (or combine) with one another, resulting in increased sonic output. By coupling the emitters to different channels, aspects of the present disclosure can further reduce the energy consumption of the catheter (such as by distributing the load across multiple channels). As a result, catheters of the present implementations can be configured to meet any given power budget. A phenomenon known as “dry firing” can occur if a spark gap “fires” or generates an electrical discharge while the fluid in the catheter is displaced away from the spark gap (such as by shock waves produced from neighboring spark gaps), which can result in failure to generate a shock wave. Because electrode pairs coupled to different channels can receive voltage pulses at different times, the fluid has more time to return to the emitters between firing of neighboring spark gaps. As a result, the risk of dry firing can be significantly reduced or mitigated by the catheters of the present implementations.

Although certain aspects of the present disclosure are described in detail herein in the context of electrohydraulic generation of shock waves, where energy is delivered via voltage or current to pairs of electrodes surrounded by a conductive solution, it should be understood that such context is provided for convenience and clarity, and the concepts disclosed herein can be applicable to any suitable energy-based lithotripsy technology. Example suitable technologies include laser generation of shock waves (where a laser pulse is transmitted into and absorbed by the medium) and piezoelectric generation of ultrasound waves (where piezoelectric transducers are used to convert electrical energy into high-frequency mechanical vibrations). These examples of energy-based lithotripsy technologies are not intended to be a comprehensive list of potential energy sources to create acoustic waves.

In some implementations, a lithotripsy catheter may be an “over-the-wire-type” (OTW) catheter. In an OTW catheter, the guidewire entry point to the catheter is at the distal end of the catheter tip, extends through the full length of the catheter through a lumen (alternatively referred to as a “guidewire lumen” and sometimes is structurally also a “central lumen”), and exits from the proximal end of the catheter via a guidewire port. In some other implementations, a lithotripsy catheter is a “rapid exchange” (Rx) catheter. An Rx design allows just a portion of the catheter to ride over a guidewire. The guidewire entry point to the catheter is at the distal end of the catheter tip, and the extends along a pathway (e.g., a dedicated guidewire lumen) through a portion of the catheter until it reaches an exit port on the side of the catheter. The guidewire exit port is generally located at a position behind (proximal from) the distal tip of the catheter, and moreover the exit port is located along the length of a catheter proximal to a functional region of the catheter (e.g., an expandable member or a tensioned member having shock wave emitters). The location of an Rx catheter guidewire exit port can vary for a given design, generally being positioned from fifteen to thirty-five centimeters (15-35 cm) behind the distal tip, although it is appreciated that in some implementations the exit port can be less than 15 cm or greater than 35 cm behind the distal tip of the catheter. Thus in use, a guidewire is delivered into the anatomy of a patient, the proximal end of the guidewire (outside the patient) is fed into the distal end opening of the catheter, and the catheter is run along the guidewire until it reaches the target tissue at the distal end of the guidewire (inside the patient); the effective difference between an OTW and an Rx catheter is where the guidewire exits the catheter. The selection between an OTW design and an Rx design is driven by factors including (but not limited to): the anatomy to be treated (e.g., coronary vasculature vs. peripheral vasculature); the required guidewire length; the trackability, stiffness, torque transmission, and deliverability of the catheter; the profile and cross-sectional dimension of the catheter, the ability to exchange a guidewire once the catheter has crossed a stenosis; the need or the ability to position the catheter’s distal end close to the guidewire tip and confirm its position.

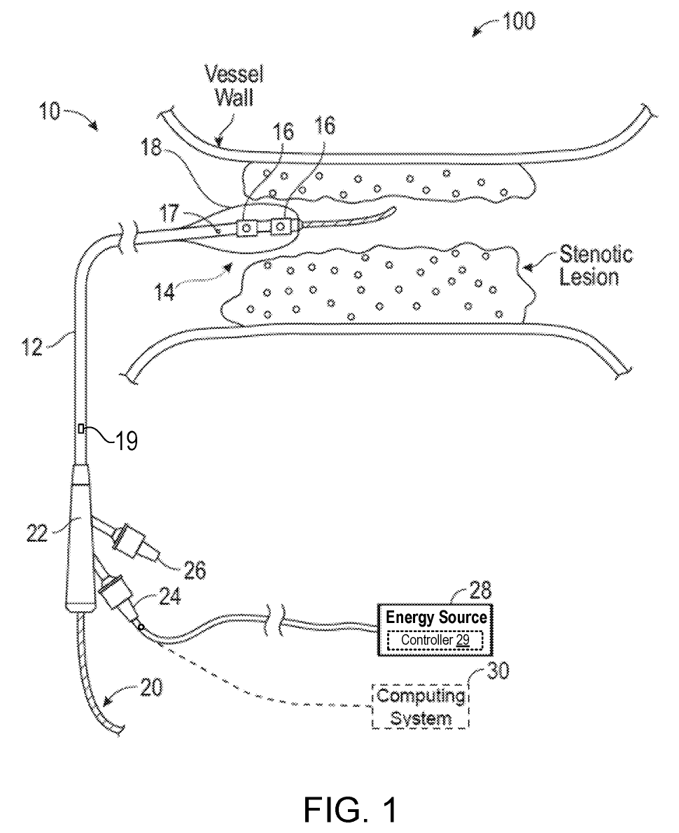

FIG. 1 shows an example medical system 100, according to some implementations. The medical system 100 can be used for lithotripsy or otherwise treating hardened lesions inside an anatomy. For example, the catheter 10 can be used to treat calcifications within blood vessels, the heart (such as inside the valves), kidneys, bladder, gallbladder, or various other anatomical locations.

The system 100 includes a catheter 10 for generating acoustic waves (such as shock waves, ultrasound waves, and/or cavitation waves) that can fragment, crack, or otherwise break up hardened lesions within the anatomy. The catheter 10 includes a proximal end 22 (which may include or form a handle) that remains outside of a patient’s vasculature during treatment. The proximal end 22 includes an entry port for receiving a guidewire 20. In some implementations, the proximal end 22 may include at least one fluid port 26 for filling and/or emptying an enclosure 18 (such as a dilating balloon) at a distal end 14 of the catheter 10. For example, the enclosure 18 can be inflated to a relatively low pressure (such as ~2–4 atm) so that the enclosure 18 expands to contact a lesion but is not an inflation pressure that substantively displaces the lesion. In some other implementations, the enclosure 18 may be omitted from the catheter 10. The proximal end 22 also includes a connection port 24 to provide an electrical or optical connection between the catheter 10 and an external energy source 28. For example, the energy source 28 may be a voltage source, a current source, a light source, or any other suitable source of energy.

A body 12 of the catheter 10 extends from its proximal end 22 to its distal end 14. The catheter body 12 can provide various internal conduits connecting elements of the distal end 14 with the proximal end 22 of the catheter 10. For example, the catheter body 12 can include an elongate tube or lumen for receiving the guidewire 20. The elongate tube may include additional lumens extending through the catheter body 12 or along an outer surface of the catheter body 12. For example, fluid lumens (such as a fluid inlet lumen and a fluid outlet lumen or a combined flush lumen) can be located along or within the catheter body 12 for carrying a fluid (or other medium) from the fluid port 26 into the enclosure 18.

The distal end 14 of the catheter 10 includes one or more emitters 16 positioned within the enclosure 18. Although two emitters 16 are shown in the example of FIG. 1, the catheter 10 can include any number of emitters. The enclosure 18 forms an annular channel around the catheter body 12 which can be filled with a medium (such as a fluid or gas) to surround the emitters 16. In some implementations, the enclosure 18 may be compliant (such as a low-profile flexible angioplasty balloon or cap, a polymer membrane in tension that can flex outward, or the like), such that the enclosure 18 expands when filled with the medium. In some other implementations, the enclosure 18 may be noncompliant (or semi-compliant), such that the enclosure 18 maintains a substantially constant volume and profile when filled with the medium.

Each emitter 16 is configured to generate acoustic waves in the medium surrounded by the enclosure 18. In some implementations, the emitters 16 may generate the acoustic waves using electrohydraulic generation techniques. In such implementations, each emitter 16 may include one or more pairs of electrodes that are separated by respective spark gaps, where the electrodes are electrically coupled to the energy source 28. In some other implementations, the emitters 16 may generate the acoustic waves using laser generation techniques. In such implementations, each emitter 16 may include one or more optical fibers that are electrically or optically coupled to the energy source 28. Still further, in some implementations, the emitters 16 may generate the acoustic waves using piezoelectric generation techniques. In such implementations, each emitter 16 may include one or more piezoelectric transducers that are electrically coupled to the energy source 28.

For electrohydraulic generation of acoustic waves, each emitter 16 includes one or more electrode pairs having first and second electrodes separated by a gap (or “spark gap”). The catheter body 12 can include one or more conductors (such as wires and/or conductive traces) for electrically coupling the emitters 16 to the energy source 28. The energy source 28 can provide energy pulses (in the form of voltage or current) to the electrode pairs to generate shock waves via the spark gaps. For example, when a voltage pulse is applied to an electrode pair, current flows across the gap between the electrodes. The medium inside the enclosure 18 can be a conductive fluid (such as saline) that facilitates the flow of current across the gap to generate shock waves. The shock waves propagate within the conductive fluid outwardly from the emitters 16 through the walls of the enclosure 18 and into a target lesion (such as the stenotic lesion depicted in FIG. 1).

The voltage pulse applied by the energy source 28 can be ~500 V–4,000 V. In some implementations, the voltage pulse applied by the voltage source can be up to 15,000 V (or higher). The pulse width of the applied voltage pulses can range between ~1–6 μs. The repetition rate or frequency of the applied voltage pulses may be between ~1–10 Hz. The total number of pulses applied by the energy source 28 can be, for example, 60 pulses, 80 pulses, 120 pulses, 300 pulses, 500 pulses, or any increments of pulses within this range. In some other implementations, the energy source 28 may be configured to deliver a packet of micro-pulses having a sub-frequency between ~100 Hz–10 kHz. The preferred voltage, repetition rate, and number of pulses may vary depending on various factors, such as the size of the lesion, the extent of calcification, the size of the blood vessel, the attributes of the patient, the stage of treatment, or various characteristics or properties of the catheter 10 and/or the emitter 16, among other factors. The magnitude of the shock waves can be controlled via the amplitude, duration, and/or repetition rate of the pulsed voltage (or current) from the energy source 28.

For laser generation of acoustic waves, the emitters 16 are configured to transmit such pulses of light into the surrounding fluid (or other medium) within the enclosure 18 based on electrical or optical energy pulses received from the energy source 28. The shock waves are created by the fluid absorbing the light emitted by the emitters 16. For example, a laser pulse can deliver a large amount of energy in a small volume of fluid, in a very short time, which creates a rapidly expanding vapor bubble that pushes the fluid outward (such as through heating and/or vaporization of the surrounding fluid). This rapid expansion produces an acoustic shock wave that propagates outward to modify the target lesion. The intensity of the acoustic energy can be higher if a fluid or other medium is chosen that exhibits strong absorption at the laser wavelength that is employed by the emitters 16.

For piezoelectric generation of acoustic waves, the emitters 16 may include piezoelectric transducers (or actuators) configured to convert the electrical pulses from the energy source 28 into mechanical vibrations in the surrounding fluid (or other medium) within the enclosure 18. A piezoelectric actuator can comprise any material or substance that physically deforms when an electric field is applied to the material. For example, a piezoelectric material expands when the applied voltage has the same polarity as its poling direction and contracts when the applied voltage has the opposite polarity as its poling direction. Thus, a piezoelectric actuator can be induced to vibrate (or cyclically expand and contract) by alternating the polarity of the voltage applied across its poles (such as by applying an alternating current (AC)). These mechanical vibrations can create acoustic pressure waves in the surrounding fluid that propagate towards the target lesion. The frequency of the acoustic pressure waves matches the frequency of the alternating voltage or current, which can be tuned to ultrasonic frequencies.

In some aspects, the energy dispersed by the emitters 16 may create one or more rapidly expanding and collapsing vapor bubbles that generate secondary shock waves (or “cavitation waves”). The enclosure 18 can mitigate thermal injury to soft tissue and reduce cavitation stresses by limiting expansion of the vapor bubbles produced during shock wave generation. For instance, the vapor bubbles hit the enclosure wall before reaching their maximum potential size, thus inducing collapse, and reducing cavitation stress and preventing soft tissue injury that can be caused by tensile stresses during cavitation bubble collapse. Once the lesions have been cracked by the acoustic shock waves, the enclosure 18 can be expanded further to increase the cross-sectional area of the lumen and improve blood flow through the lumen. In some implementations, the conductive fluid may contain x-ray contrast fluid for fluoroscopic viewing of the catheter 10 during use.

In some implementations, the catheter 10 may include one or more sensors 17 positioned at various locations on the catheter 10 (including the distal end 14, the proximal end 22, or any location along the catheter body 12). For example, one or more sensors 17 can be positioned proximate to one or more of the emitters 16. The sensors 17 can include any suitable sensor devices, such as a position sensor, a pressure sensor, a thermal sensor, an electrical sensor (e.g., current, voltage, resistance, and/or impedance sensors), or a visualization element, among other examples. Measurements from sensors 17 can be used to provide feedback about the catheter 10 and/or medical procedure. In some implementations, the feedback may be displayed or otherwise presented to a user or operator of the medical system 10, for example, via an interface or display of the computing system 30 (not shown for simplicity). Example feedback about the catheter 10 can include, among other examples, a temperature or pressure inside the enclosure 18, a voltage or current supplied to the emitters 16, a position and/or orientation of the catheter 10 inside an anatomy, and/or a path traversed by the catheter 10. Example feedback about the procedure can include guidance on further steps to be performed using the catheter 10 (such as how to move or position the catheter 10).

For example, where the sensors 17 include a pressure sensor, a slight decrease in pressure can be detected when the enclosure 18 is able to further expand without changing the volume of fluid therein. Feedback associated with such decreases in pressure may signal a successful cracking of calcified lesions and/or guide the user to move the catheter 10 to treat another region of a lesion. A significant decrease in pressure can be detected when the enclosure 18 has lost its seal with the catheter body 12 and/or fluid therein. Feedback associated with such decreases in pressure may signal a rupture or failure of the enclosure 18 and/or guide the user to retract the catheter 10. Where the sensors 17 include a visualization element (such as a camera, optical coherence tomography (OCT), or ultrasound imaging device), the feedback may include real-time images or video depicting a scene proximate the catheter 10. Such feedback can enable a user to visualize or better understand the spatial relationship between the catheter 10 and the target lesion or anatomy at any given time. In some implementations, the medium held by the enclosure 18 may contain x-ray contrast fluid for fluoroscopic viewing of the catheter 10.

In some implementations, the catheter 10 may include a memory 19 disposed thereon that stores information about the catheter 10 (such as a “part number” or other identifier) and/or parameters for receiving energy from the energy source 28 (also referred to as “configuration parameters”). The memory 19 can be any type of non-volatile memory, such as erasable programmable read-only memory (EPROM) or electrically erasable programmable read-only memory (EEPROM), among other examples. Example configuration parameters that can be stored in the memory 19 include a maximum number of energy pulses that the catheter 10 can receive until it reaches its end of life (such as when the emitters 16 wear out or are otherwise unable to produce acoustic waves suitable for treatment of calcified lesions) and a total number of energy pulses received by the catheter 10 during its lifetime.

A controller 29 associated with the energy source 28 can read the information stored in the memory 19 when the catheter 10 is connected or otherwise coupled to the energy source 28 (such as via the connection port 24) and configure the energy source 28 based on the retrieved information. For example, the controller 29 may determine the type of catheter 10 coupled to the energy source 28 (including a number of emitters 16 and/or size of the enclosure 18) based on the part number and/or other information stored in the memory 19. The controller 29 also may determine a number of energy pulses remaining over the lifetime of the catheter 10 and display the number on a graphical interface (not shown for simplicity) while counting down to the catheter’s end of life. In some implementations, the controller 29 may further control various aspects of energy delivery from the energy source 28 to the catheter 10 for generating acoustic waves at the emitters 16.

In some implementations, the catheter 10 may include fewer or more emitters 16 than those depicted in FIG. 1. For example, catheters with more emitters can be used to treat longer lesions and/or multiple lesions in a shorter amount of time (such as by reducing the amount of moving or repositioning of the catheter). However, the strength and/or efficacy of the acoustic waves generally depends on the amount of energy (or voltage) that can be supplied to each acoustic wave transducer, which may be limited by the energy source 28. For example, some existing energy sources may effectively produce at most four shock waves (via the two emitters 16) with a single energy pulse. Some other example energy sources may effectively produce up to six shock waves with a single energy pulse.

Aspects of the present disclosure recognize that emitters can be added to a catheter, without increasing its energy consumption, by supplying energy to the emitters via different channels. The emitters can be coupled to the channels so that the number of acoustic wave transducers receiving the same energy pulse does not exceed the capabilities of the energy source. With reference for example to FIG. 1, the emitter 16 closest to the distal tip of the catheter 10 (also referred to as the “distal emitter”) can be coupled to a first channel and the emitter 16 closer to the proximal end 22 (also referred to as the “proximal emitter”) can be coupled to a second channel. The energy source 28 may output a series of energy pulses on the first and second channels in an interleaved fashion, causing the distal emitter 16 to produce acoustic waves at different times than the proximal emitter 16. Because only half the emitters 16 produce acoustic waves in response to each energy pulse, the power consumption of the catheter 10 is reduced while maintaining the same acoustic coverage (compared to catheters having two emitters coupled to the same channel).

To optimize the sonic output of the catheter 10 for the available power budget of the energy source 28, one or more emitters can be added to the catheter 10. In some aspects, at least one of the emitters may be coupled to multiple channels. With reference for example to FIG. 1, a third emitter (also referred to as a “shared emitter”) can be inserted between the distal emitter 16 and the proximal emitter 16 along the length of the catheter body 12. The shared emitter can include one or more acoustic wave transducers coupled to the first channel and one or more acoustic wave transducers coupled to the second channel so that the shared emitter can generate acoustic waves (albeit via different pairs of electrodes) in response to each energy pulse output by the energy source 28. The acoustic waves generated by the shared emitter can constructively interfere (or combine) with acoustic waves generated by the distal emitter 16 and the proximal emitter 16, thereby increasing the sonic output of the catheter 10.

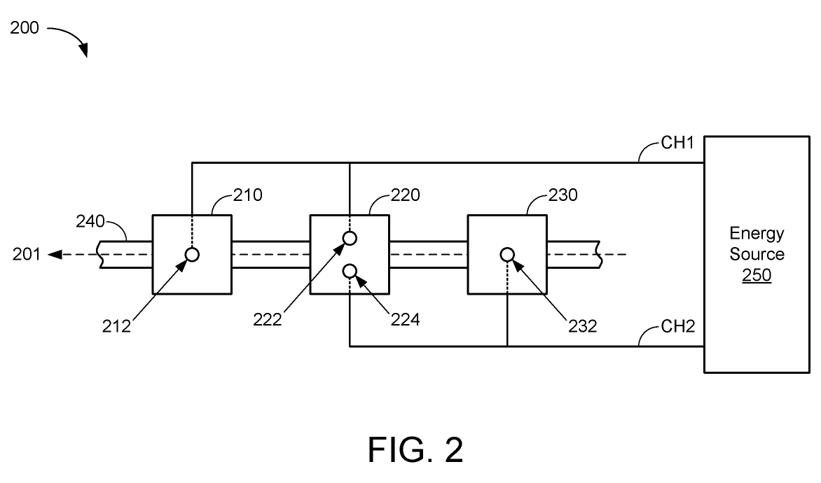

FIG. 2 shows a block diagram of an example catheter system 200, according to some implementations. More specifically, FIG. 2 shows a distal portion of a catheter having multiple emitters 210, 220, and 230 coupled to an elongate shaft 240. In some implementations, the elongate shaft 240 may be one example of the catheter body 12 of FIG. 1. Although three emitters 210, 220, and 230 are shown coupled to two channels CH1 and CH2 in the example of FIG. 2, the catheter system 200 can include any number (X) of emitters coupled to any number (Y) of channels.

As shown in FIG. 2, the emitters 210, 220, and 230 are positioned at different locations (e.g., longitudinally) along an axis 201 of the shaft 240. Each emitter includes one or more acoustic wave transducers (such as electrode pairs forming respective spark gaps). For example, the first emitter 210 is shown to include a transducer 212, the second emitter 220 is shown to include transducers 222 and 224, and the third emitter 230 is shown to include a transducer 232. More generally, a catheter can include any number (N) of transducers distributed in any manner across the X emitters. In some implementations, the emitters nearest the distal and proximal ends of the shaft 240 (such as the emitters 210 and 230) may include half as many transducers as any emitters therebetween (such as the emitter 220). In some aspects, the transducers 212, 222, 224, and 232 may be surrounded by a medium (such as a fluid or gas) sealed within an enclosure (such as the enclosure 18 of FIG. 1).

In some implementations, the first emitter 210 may be coupled to an energy source 250 (such as a voltage source, a current source, or any other suitable source of energy) via a first channel (CH1), the third emitter 230 may be coupled to the energy source 250 via a second channel (CH2), and the second emitter 220 may be coupled to the energy source 250 via both channels CH1 and CH2. Thus, the second emitter 220 also may be referred to as a “shared emitter.” More specifically, the transducers 212 and 222 of the emitters 210 and 220, respectively, are coupled to the first channel CH1, whereas the transducers 224 and 232 of the emitters 220 and 230, respectively, are coupled to the second channel CH2. In some implementations, the channels CH1 and CH2 may be formed by one or more conductors (such as wires and/or conductive traces) that can carry current (or voltage) to the transducers 212, 222, 224, and 232. Thus, each of the channels CH1 and CH2 can include an output path and a return path for the current (not shown for simplicity).

The energy source 250 is configured to output a series of energy pulses on the channels CH1 and CH2 that can produce shock waves via the transducers 212, 222, 224, and 232. For example, the transducers 212 and 222 may generate shock waves in response to energy pulses on the first channel CH1 and the transducers 224 and 232 may generate shock waves in response to energy pulses on the second channel CH2. In some implementations, the energy source 250 may output the series of energy pulses on the channels CH1 and CH2 in an interleaved manner so that the transducers 212 and 222 produce shock waves at different times than the transducers 224 and 232.

In this way, the second emitter 220 can generate shock waves in response to each energy pulse output by the energy source 250, whereas each of the first emitter 210 and the third emitter 230 can generate shock waves in response to every other energy pulse output by the energy source 250. In other words, the emitter 220 produces shock waves twice as often as any of the emitters 210 or 230. The shock waves produced by the second emitter 220 can constructively interfere with shock waves produced by the first emitter 210 and the third emitter 230, thereby increasing the sonic output of the catheter system 200.

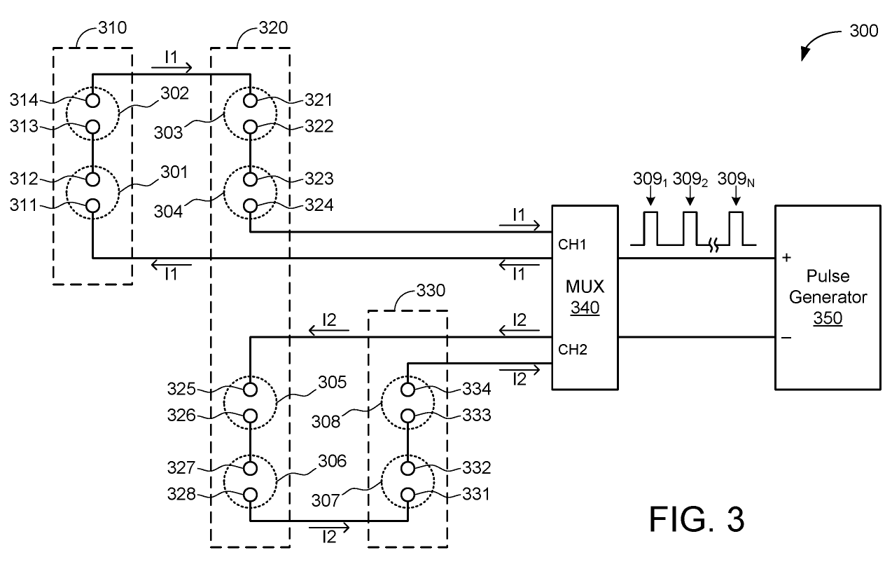

FIG. 3 shows a circuit diagram of an example catheter system 300, according to some implementations. In some implementations, the catheter system 300 may be one example of the catheter system 200 of FIG. 2. More specifically, FIG. 3 shows a series of emitters 310, 320, and 330 that can be disposed on the distal portion of an elongate shaft (such as the shaft 240 of FIG. 2). In some implementations, the emitters 310, 320, and 330 may be examples of the emitters 210, 220, and 230, respectively, of FIG. 2.

The first emitter 310 includes a first pair of electrodes 311 and 312 forming a first spark gap 301 and a second pair of electrodes 313 and 314 forming a second spark gap 302. The electrode 312 is coupled to the electrode 313, creating a path for current to flow between the first spark gap 301 and the second spark gap 302. In some implementations, the electrodes 312 and 313 may be formed by a single conductive element (also referred to as a “common electrode”) that is shared between the spark gaps 301 and 302.

The second emitter 320 (also referred to as the “shared emitter”) includes a first pair of electrodes 321 and 322 forming a third spark gap 303, a second pair of electrodes 323 and 324 forming a fourth spark gap 304, a third pair of electrodes 325 and 326 forming a fifth spark gap 305, and a fourth pair of electrodes 327 and 328 forming a sixth spark gap 306. The electrode 322 is coupled to the electrode 323, creating a path of current between the third spark gap 303 and the fourth spark gap 304. The electrode 326 is coupled to the electrode 327, creating a path for current to flow between the fifth spark gap 305 and the sixth spark gap 306.

In some implementations, the electrodes 322 and 323 may be formed by a single common electrode that is shared between the spark gaps 303 and 304. In some implementations, the electrodes 326 and 327 may be formed by a single common electrode that is shared between the spark gaps 305 and 306. In some other implementations, the electrodes 322, 323, 326, and 327 may be formed by a single common electrode that is shared between the spark gaps 303, 304, 305, and 306.

The third emitter 330 includes a first pair of electrodes 331 and 332 forming a seventh spark gap 307 and a second pair of electrodes 333 and 334 forming an eighth spark gap 308. The electrode 332 is coupled to the electrode 333, creating a path for current to flow between the seventh spark gap 307 and the eighth spark gap 308. In some implementations, the electrodes 332 and 333 may be formed by a single common electrode that is shared between the spark gaps 307 and 308.

As shown in FIG. 3, the first emitter 310 is coupled to a pulse generator 350 via a first conductive channel (CH1), the third emitter 330 is coupled to the pulse generator 350 via a second conductive channel (CH2), and the second emitter 320 is coupled to the pulse generator 350 via both conductive channels CH1 and CH2. The conductive channels CH1 and CH2 are electrically isolated from one another. In some implementations, the channels CH1 and CH2 may be examples of the channels CH1 and CH2, respectively, of FIG. 2.

In the example of FIG. 3, the spark gaps 301–304 are coupled to the first channel CH1 and the spark gaps 305–308 are coupled to the second channel CH2. The electrode 314 is coupled to the electrode 321, creating a path for current to flow between the emitters 310 and 320. The electrode 328 is coupled to the electrode 331, creating a path for current to flow between the emitters 320 and 330.

The pulse generator 350 is configured to output a series of voltage pulses 309₁–309_{N} that can produce shock waves via the spark gaps 301–308. In some implementations, the catheter system 300 may include a multiplexer (MUX) 340 to distribute the voltage pulses 309₁–309_{N} between the conductive channels CH1 and CH2. In some implementations, the multiplexer 340 may distribute the voltage pulses 309₁–309_{N} in an interleaved manner so that odd-numbered pulses (such as 309₁) are driven onto the first conductive channel CH1 and even-numbered pulses (such as 309₂) are driven onto the second conductive channel CH2. Although the MUX 340 and the pulse generator 350 are depicted as separate and discrete components in FIG. 3, the MUX 340 can also be combined or otherwise integrated with the pulse generator 350.

The voltage pulses driven onto the first conductive channel CH1 cause a current (I1) to flow through the first emitter 310 and the second emitter 320. As shown by the arrows in FIG. 3, the current I1 initially enters the first emitter 310 via the electrode 311. Within the first emitter 310, the current I1 jumps the first spark gap 301, between the electrodes 311 and 312, and subsequently jumps the second spark gap 302 between the electrodes 313 and 314. Each jump of the current I1, across the spark gaps 301 and 302, creates a spark that gives rise to a respective shock wave.

As further shown by the arrows in FIG. 3, the current I1 exits the first emitter 310 via the electrode 314 and enters the second emitter 320 via the electrode 321. Within the second emitter 320, the current I1 jumps the third spark gap 303, between the electrodes 321 and 322, and subsequently jumps the fourth spark gap 304 between the electrodes 323 and 324. Each jump of the current I1, across the spark gaps 303 and 304, creates a spark that gives rise to a respective shock wave. The current I1 exits the second emitter 320 via the electrode 324 and returns to the pulse generator 305 (or ground).

The voltage pulses driven onto the second conductive channel CH2 cause a current (I2) to flow through the second emitter 320 and the third emitter 330. As shown by the arrows in FIG. 3, the current I2 initially enters the second emitter 320 via the electrode 325. Within the second emitter 320, the current I2 jumps the fifth spark gap 305, between the electrodes 325 and 326, and subsequently jumps the sixth spark gap 306 between the electrodes 327 and 328. Each jump of the current I2, across the spark gaps 305 and 306, creates a spark that gives rise to a respective shock wave.

As further shown by the arrows in FIG. 3, the current I2 exits the second emitter 320 via the electrode 328 and enters the third emitter 330 via the electrode 331. Within the third emitter 330, the current I2 jumps the seventh spark gap 307, between the electrodes 331 and 332, and subsequently jumps the eighth spark gap 308 between the electrodes 333 and 334. Each jump of the current I2, across the spark gaps 307 and 308, creates a spark that gives rise to a respective shock wave. The current I2 exits the third emitter 330 via the electrode 334 and returns to the pulse generator 350 (or ground).

In some aspects, each of the emitters 310, 320, and 330 may be configured to wrap around an outer diameter (or circumference) of the elongate shaft. For example, the spark gaps 301–308 may be positioned radially around an axis of the shaft so that the shock waves radiate outward, away from the axis of the shaft. In some implementations, the electrodes may be layered on top of one another so that at least one electrode in each electrode pair is positioned closer to the outer diameter of the shaft (such as in a radial direction) and the other electrode in the electrode pair is positioned further away from the outer diameter of the shaft.

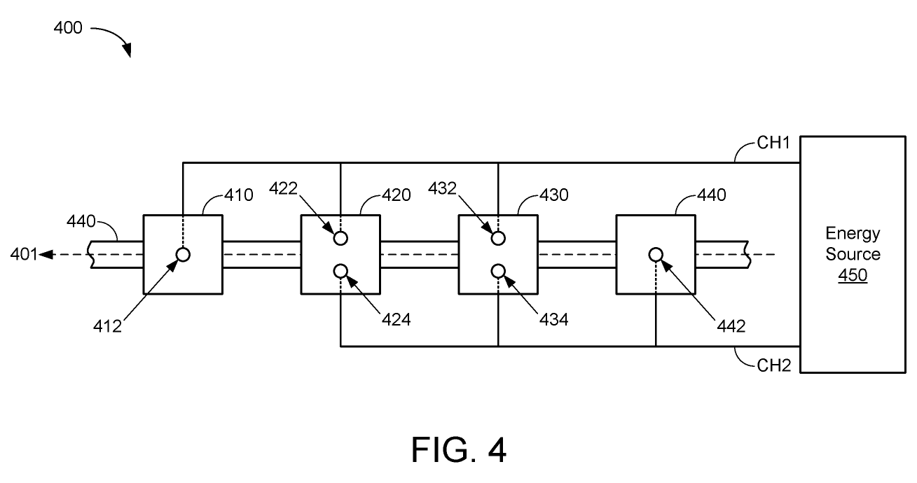

FIG. 4 shows another block diagram of an example catheter system 400, according to some implementations. More specifically, FIG. 4 shows a distal portion of a catheter having multiple emitters 410, 420, 430, and 440 coupled to an elongate shaft 440. In some implementations, the elongate shaft 440 may be one example of the catheter body 12 of FIG. 1. Although four emitters 410, 420, 430, and 440 are shown coupled to two channels CH1 and CH2 in the example of FIG. 4, the catheter system 400 can include any number (X) of emitters coupled to any number (Y) of channels.

As shown in FIG. 4, the emitters 410, 420, 430, and 440 are positioned at different locations (e.g., longitudinally) along an axis 401 of the shaft 440. Each emitter includes one or more acoustic wave transducers (such as electrode pairs forming respective spark gaps). For example, the first emitter 410 is shown to include a transducer 412, the second emitter 420 is shown to include transducers 422 and 424, the third emitter 430 is shown to include transducers 432 and 434, and the fourth emitter 440 is shown to include a transducer 442. As described with reference to FIG. 2, a catheter can include any number (N) of transducers distributed in any manner across the X emitters. In some implementations, the emitters nearest the distal and proximal ends of the shaft 440 (such as the emitters 410 and 440) may include half as many transducers as any emitters therebetween (such as the emitters 420 and 430). In some aspects, the transducers 412, 422, 424, 432, 434, and 442 may be surrounded by a medium (such as a fluid or gas) sealed within an enclosure (such as the enclosure 18 of FIG. 1).

In some implementations, the first emitter 410 may be coupled to an energy source 450 (such as a voltage source, a current source, or any other suitable source of energy) via a first channel (CH1), the fourth emitter 440 may be coupled to the energy source 450 via a second channel (CH2), and the second and third emitters 420 and 430 may be coupled to the energy source 450 via both channels CH1 and CH2. Thus, the second and third emitters 420 and 430 are the shared emitters. More specifically, the transducers 412, 422, and 432 of the emitters 410, 420, and 430, respectively, are coupled to the first channel CH1, whereas the transducers 424, 434, and 442 of the emitters 420, 430, and 440, respectively, are coupled to the second channel CH2. In some implementations, the channels CH1 and CH2 may be formed by one or more conductors (such as wires and/or conductive traces) that can carry current (or voltage) to the transducers 412, 422, 424, 432, 434, and 442. Thus, each of the channels CH1 and CH2 can include an output path and a return path for the current (not shown for simplicity).

The energy source 450 is configured to output a series of energy pulses on the channels CH1 and CH2 that can produce shock waves via the transducers 412, 422, 424, 432, 434, and 442. For example, the transducers 412, 422, and 432 may generate shock waves in response to energy pulses on the first channel CH1, and the transducers 424, 434, and 442 may generate shock waves in response to energy pulses on the second channel CH2. In some implementations, the energy source 450 may output the series of energy pulses on the channels CH1 and CH2 in an interleaved manner so that the transducers 412, 422, and 432 produce shock waves at different times than the transducers 424, 434, and 442.

In this way, the second and third emitters 420 and 430 can generate shock waves in response to each energy pulse output by the energy source 450, whereas each of the first emitter 410 and the fourth emitter 440 can generate shock waves in response to every other energy pulse output by the energy source 450. In other words, the emitters 420 and 430 produce shock waves twice as often as any of the emitters 410 or 440. The shock waves produced by the second and third emitters 420 and 430 can constructively interfere with shock waves produced by the first emitter 410 and the fourth emitter 440, thereby increasing the sonic output of the catheter system 400.

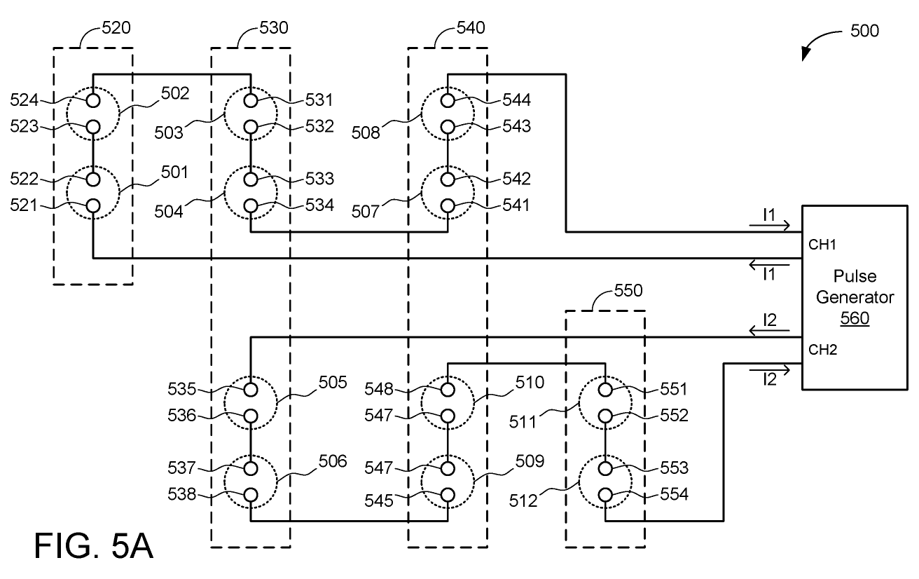

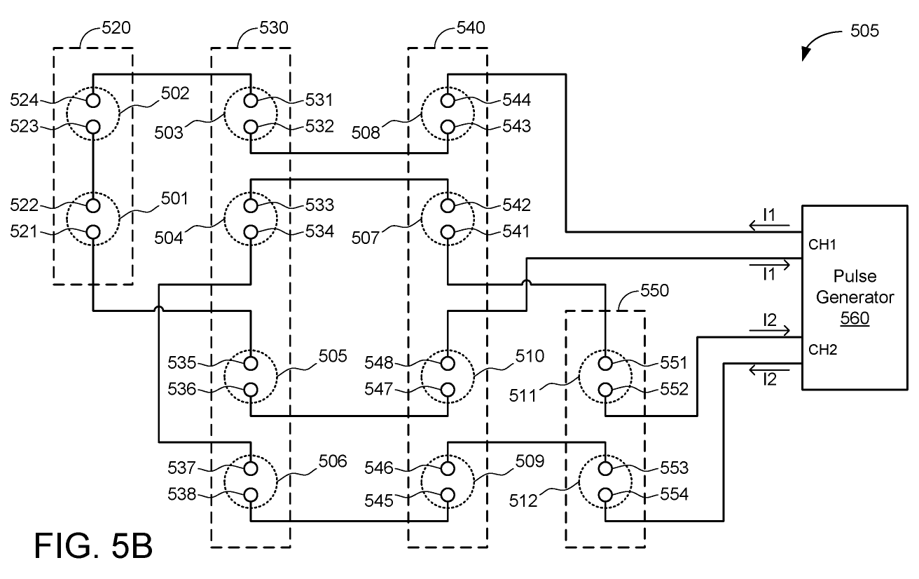

FIG. 5A shows another circuit diagram of an example catheter system 500, according to some implementations. In some implementations, the catheter system 500 may be one example of the catheter system 400 of FIG. 4. More specifically, FIG. 5A shows a series of emitters 520, 530, 540, and 550 that can be disposed on the distal portion of an elongate shaft (such as the shaft 440 of FIG. 4). In some implementations, the emitters 520, 530, 540, and 550 may be examples of the emitters 410, 420, 430, and 440, respectively, of FIG. 4.

The first emitter 520 includes a first pair of electrodes 521 and 522 forming a first spark gap 501 and a second pair of electrodes 523 and 524 forming a second spark gap 502. The electrode 522 is coupled to the electrode 523, creating a path for current to flow between the first spark gap 501 and the second spark gap 502. In some implementations, the electrodes 522 and 523 may be formed by a single conductive element (such as a common electrode) that is shared between the spark gaps 501 and 502.

The second emitter 530 includes a first pair of electrodes 531 and 532 forming a third spark gap 503, a second pair of electrodes 533 and 534 forming a fourth spark gap 504, a third pair of electrodes 535 and 536 forming a fifth spark gap 505, and a fourth pair of electrodes 537 and 538 forming a sixth spark gap 506. The electrode 532 is coupled to the electrode 533, creating a path of current between the third spark gap 503 and the fourth spark gap 504. The electrode 536 is coupled to the electrode 537, creating a path for current to flow between the fifth spark gap 505 and the sixth spark gap 506.

In some implementations, the electrodes 532 and 533 may be formed by a single common electrode that is shared between the spark gaps 503 and 504. In some implementations, the electrodes 536 and 537 may be formed by a single common electrode that is shared between the spark gaps 505 and 506. In some other implementations, the electrodes 532, 533, 536, and 537 may be formed by a single common electrode that is shared between the spark gaps 503, 504, 505, and 506.

The third emitter 540 includes a first pair of electrodes 541 and 542 forming a seventh spark gap 507, a second pair of electrodes 543 and 544 forming an eighth spark gap 508, a third pair of electrodes 545 and 546 forming a ninth spark gap 509, and a fourth pair of electrodes 547 and 548 forming a tenth spark gap 510. The electrode 542 is coupled to the electrode 543, creating a path of current between the seventh spark gap 507 and the eighth spark gap 508. The electrode 546 is coupled to the electrode 547, creating a path for current to flow between the ninth spark gap 509 and the tenth spark gap 510.

In some implementations, the electrodes 542 and 543 may be formed by a single common electrode that is shared between the spark gaps 507 and 508. In some implementations, the electrodes 546 and 547 may be formed by a single common electrode that is shared between the spark gaps 509 and 510. In some other implementations, the electrodes 542, 543, 546, and 547 may be formed by a single common electrode that is shared between the spark gaps 507, 508, 509, and 510.

The fourth emitter 550 includes a first pair of electrodes 551 and 552 forming an eleventh spark gap 511 and a second pair of electrodes 553 and 554 forming a twelfth spark gap 512. The electrode 552 is coupled to the electrode 553, creating a path for current to flow between the eleventh spark gap 511 and the twelfth spark gap 512. In some implementations, the electrodes 552 and 553 may be formed by a single common electrode that is shared between the spark gaps 511 and 512.

As shown in FIG. 5A, the first emitter 520 is coupled to a pulse generator 560 via a first conductive channel (CH1), the fourth emitter 550 is coupled to the pulse generator 560 via a second conductive channel (CH2), and each of the second and third emitters 530 and 540 is coupled to the pulse generator 560 via both conductive channels CH1 and CH2. The conductive channels CH1 and CH2 are electrically isolated from one another. In some implementations, the channels CH1 and CH2 may be examples of the channels CH1 and CH2, respectively, of FIG. 4.

In the example of FIG. 5A, the spark gaps 501–504, 507, and 508 are coupled to the first channel CH1, and the spark gaps 505, 506, and 509–512 are coupled to the second channel CH2. The electrode 524 is coupled to the electrode 531, creating a path for current to flow between the emitters 520 and 530. The electrode 538 is coupled to the electrode 545, and the electrode 534 is coupled to the electrode 541, creating paths for current to flow between the emitters 530 and 540. The electrode 548 is coupled to the electrode 551, creating a path for current to flow between the emitters 540 and 550.

The pulse generator 560 is configured to output a series of voltage pulses that can produce shock waves via the spark gaps 501–512 (such as described with reference to FIG. 3). In some implementations, the catheter system 500 may include a multiplexer (not shown for simplicity) to distribute the voltage pulses between the conductive channels CH1 and CH2. As described with reference to FIG. 3, the multiplexer can be separate from, or integrated with, the pulse generator 560. In some implementations, the pulse generator 560 (or multiplexer) may distribute the voltage pulses in an interleaved manner so that odd-numbered pulses are driven onto the first conductive channel CH1 and even-numbered pulses are driven onto the second conductive channel CH2.

The voltage pulses driven onto the first conductive channel CH1 cause a current (I1) to flow through the emitters 520, 530, and 540. The current I1 initially enters the first emitter 520 via the electrode 521. Within the first emitter 520, the current I1 jumps the first spark gap 501, between the electrodes 521 and 522, and subsequently jumps the second spark gap 502 between the electrodes 523 and 524. Each jump of the current I1, across the spark gaps 501 and 502, creates a spark that gives rise to a respective shock wave.

The current I1 exits the first emitter 520 via the electrode 524 and enters the second emitter 530 via the electrode 531. Within the second emitter 530, the current I1 jumps the third spark gap 503, between the electrodes 531 and 532, and subsequently jumps the fourth spark gap 504 between the electrodes 533 and 534. Each jump of the current I1, across the spark gaps 503 and 504, creates a spark that gives rise to a respective shock wave.

The current I1 exits the second emitter 530 via the electrode 534 and enters the third emitter 540 via the electrode 541. Within the third emitter 540, the current I1 jumps the seventh spark gap 507, between the electrodes 541 and 542, and subsequently jumps the eighth spark gap 508 between the electrodes 543 and 544. Each jump of the current I1, across the spark gaps 507 and 508, creates a spark that gives rise to a respective shock wave. The current I1 exits the third emitter 550 via the electrode 554 and returns to the pulse generator 560 (or ground).

The voltage pulses driven onto the second conductive channel CH2 cause a current (I2) to flow through the emitters 530, 540, and 550. The current I2 initially enters the second emitter 530 via the electrode 535. Within the second emitter 530, the current I2 jumps the fifth spark gap 505, between the electrodes 535 and 536, and subsequently jumps the sixth spark gap 506 between the electrodes 537 and 538. Each jump of the current I2, across the spark gaps 505 and 506, creates a spark that gives rise to a respective shock wave.

The current I2 exits the second emitter 530 via the electrode 538 and enters the third emitter 540 via the electrode 545. Within the third emitter 534, the current I2 jumps the ninth spark gap 509, between the electrodes 545 and 546, and subsequently jumps the tenth spark gap 510 between the electrodes 547 and 548. Each jump of the current I2, across the spark gaps 509 and 510, creates a spark that gives rise to a respective shock wave.

The current I2 exits the third emitter 540 via the electrode 548 and enters the fourth emitter 550 via the electrode 551. Within the fourth emitter 550, the current I2 jumps the eleventh spark gap 511, between the electrodes 551 and 552, and subsequently jumps the twelfth spark gap 512 between the electrodes 553 and 554. Each jump of the current I2, across the spark gaps 511 and 512, creates a spark that gives rise to a respective shock wave. The current I2 exits the fourth emitter 550 via the electrode 554 and returns to the pulse generator 560 (or ground).

FIG. 5B shows another circuit diagram of an example catheter system 505, according to some implementations. More specifically, FIG. 5B depicts an alternative coupling between the emitters 520, 530, 540, and 550 and the conductive channels CH1 and CH2. As shown in FIG. 5B, the first emitter 520 is coupled to a pulse generator 560 via the first conductive channel CH1, the fourth emitter 550 is coupled to the pulse generator 560 via the second conductive channel CH2, and each of the second and third emitters 530 and 540 is coupled to the pulse generator 560 via both conductive channels CH1 and CH2.

In the example of FIG. 5B, the spark gaps 501–503, 505, 508, and 510 are coupled to the first channel CH1, and the spark gaps 504, 506, 507, 509, 511, and 512 are coupled to the second channel CH2. The electrode 524 is coupled to the electrode 531, and the electrode 521 is coupled to the electrode 535, creating paths for current to flow between the emitters 520 and 530. The electrode 532 is coupled to the electrode 543, the electrode 533 is coupled to the electrode 542, the electrode 536 is coupled to the electrode 547, and the electrode 538 is coupled to the electrode 545, creating paths for current to flow between the emitters 530 and 540. The electrode 541 is coupled to the electrode 551, and the electrode 546 is coupled to the electrode 553, creating paths for current to flow between the emitters 540 and 550.

The voltage pulses driven onto the first conductive channel CH1 cause a current (I1) to flow through the emitters 520, 530, and 540. The current I1 initially enters the third emitter 540 via the electrode 544. Within the third emitter 540, the current I1 jumps the eighth spark gap 508, between the electrodes 544 and 543.

The current I1 exits the third emitter 540 via the electrode 543 and enters the second emitter 530 via the electrode 532. Within the second emitter 530, the current I1 jumps the third spark gap 503, between the electrodes 532 and 531.

The current I1 exits the second emitter 530 via the electrode 531 and enters the first emitter 520 via the electrode 524. Within the first emitter 520, the current I1 jumps the second spark gap 502, between the electrodes 524 and 523, and subsequently jumps the first spark gap 501 between the electrodes 522 and 521.

The current I1 exits the first emitter 520 via the electrode 521 and enters the second emitter 530 via the electrode 535. Within the second emitter 530, ethe current I1 jumps the fifth spark gap 505, between the electrodes 535 and 536.

The current I1 exits the second emitter 530 via the electrode 53 and enters the third emitter 540 via the electrode 547. Within the third emitter 540, the current I1 jumps the tenth spark gap 510, between the electrodes 547 and 548. The current I1 exits the third emitter 540 via the electrode 548 and returns to the pulse generator 560 (or ground).

The voltage pulses driven onto the second conductive channel CH2 cause a current (I2) to flow through the emitters 530, 540, and 550. The current I2 initially enters the fourth emitter 550 via the electrode 554. Within the fourth emitter 550, the current I2 jumps the twelfth spark gap 512, between the electrodes 554 and 553.

The current I2 exits the fourth emitter 550 via the electrode 553 and enters the third emitter 540 via the electrode 546. Within the third emitter 540, the current I2 jumps the ninth spark gap 509, between the electrodes 546 and 545.

The current exits the third emitter 540 via the electrode 545 and enters the second emitter 530 via the spark gap 538. Within the second emitter 530, the current I2 jumps the sixth spark gap 506, between the electrodes 538 and 537, and subsequently jumps the fourth spark gap 504 between the electrodes 533 and 534.

The current I2 exits the second emitter 530 via the electrode 533 and enters the third emitter 540 via the electrode 542. Within the third emitter 540, the current I2 jumps the seventh spark gap 507, between the electrodes 542 and 541.

The current I2 exits the third emitter 540 via the electrode 541 and enters the fourth emitter 550 via the electrode 551. Within the fourth emitter 550, the current I2 jumps the eleventh spark gap 511, between the electrodes 551 and 552. The current I2 exits the fourth emitter 550 via the electrode 552 and returns to the pulse generator 560 (or ground).

In some aspects, each of the emitters 520, 530, 540, and 550 may be configured to wrap around an outer diameter (or circumference) of the elongate shaft. For example, the spark gaps 501–512 may be positioned radially around an axis of the shaft so that the shock waves radiate outward, away from the axis of the shaft. In some implementations, the electrodes may be layered on top of one another so that at least one electrode in each electrode pair is positioned closer to the outer diameter of the shaft (such as in a radial direction) and the other electrode in the electrode pair is positioned further away from the outer diameter of the shaft.

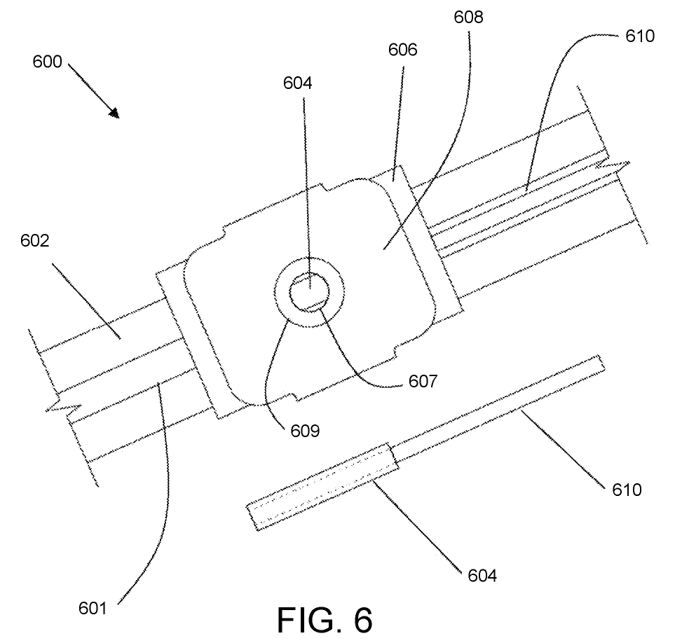

FIG. 6 shows an example emitter assembly 600, according to some implementations. In some implementations, the emitter assembly 600 may be one example of any of the emitters 310, 320, 330, 520, 530, 540 or 550 of FIGS. 3, 5A, and 5B. More specifically, the emitter assembly 600 includes an arrangement of electrodes forming spark gaps that can generate shock waves in response to voltage pulses applied thereto.

In the example of FIG. 6, the emitter assembly 600 is shown to include an inner electrode 604, an insulating layer or sheath 606 disposed over the inner electrode 604 and circumferentially wrapped around an elongate member 602, and an outer electrode sheath 608 disposed over the insulating sheath 606. In some implementations, the elongate member 602 may be one example of the elongate shaft 240 of FIG. 2. The emitter assembly 600 can be bonded to the shaft 602, for example, using an adhesive (such as glue) or any other suitable bonding agent. While the insulating sheath 606 is depicted as fully circumscribing the elongate member 602, in some other implementations, an insulating layer may not fully circumscribe the elongate member 602 and may instead be disposed over certain portions of the elongate member 602. The insulating sheath 606 has an opening 607 that is coaxially aligned over the inner electrode 604, and the outer electrode sheath 608 has an opening 609 that is coaxially aligned over the opening 607 of the insulating sheath 606.

In some implementations, the emitter assembly 600 may be rotationally symmetric about the axis of the elongate member 602. In other words, one or more sides of the emitter assembly 600 (hidden from view in FIG. 6) may mirror what is shown in FIG. 6. For example, the emitter assembly 600 may include a second inner electrode that is circumferentially opposite the inner electrode 604 (or separated by 180 degrees around the axis of the elongate member 602). The insulating sheath 606 may have a second opening that is coaxially aligned over the second inner electrode, and the outer electrode sheath 608 may have a second opening that is coaxially aligned over the second opening of the insulating sheath 606. In such implementations, the outer electrode sheath 608 may serve as a common electrode that is paired with both the inner electrode 604 and the second inner electrode (on the other side of the elongate member 602).

The inner electrode 604 and the outer electrode sheath 608 form a first spark gap (across the openings 607 and 609) that can generate shock waves which propagate radially in a first direction perpendicular to the axis of the elongate member 602. Similarly, the second inner electrode and the outer electrode sheath 608 form a second spark gap (across the openings coaxial with the second inner electrode) that can generate shock waves which propagate radially in a second direction opposite the first direction. The diameter of the openings in the outer electrode sheath 608 may be larger than the diameter of the openings in the insulating sheath 606. The size and/or ratio of the openings in the outer electrode sheath 608 and the insulating sheath 606 can affect one or more characteristics of the shock waves. Similarly, the thickness and/or material of the insulating layer 606 may depend on the magnitude of the voltage to be applied across the electrodes.

The inner electrode 604 can be electrically coupled to a pulse generator (such as any of the pulse generators 350 or 560 of FIGS. 3, 5A and 5B, via one or more wires 610 and/or conductive traces disposed within a longitudinal groove 601 along an outer surface of the elongate member 602. The wires 610 may be electrically insulated along its length except for one or more regions where the electrically conductive core of the wire 610 is exposed to contact a portion of the inner electrode 604. The wires 610 can be made of any conductive material such as, for example, free oxygen copper or copper or silver. In some implementations, the inner electrode 604 may be a hypotube that is crimped over the distal tip of the wires 610, where the wires 610 are enclosed or encapsulated within the groove 601 of the elongate member 602. The hypotube can be made of stainless steel, tungsten, a platinum-iridium alloy, or any other material with similar hardness.

In some implementations, the outer electrode sheath 608 may be a cylindrical band that circumscribes the outer diameter of the elongate member 602. In some other implementations, the outer electrode sheath 608 may be a curved band (such as in the shape of a “C”) that only partially extends around the outer diameter of the elongate member 602. Using a C-shaped band for the outer electrode sheath 608 can improve the flexibility of the catheter, making it easier to navigate within an anatomical lumen. Additionally, the use of C-shaped bands can reduce the crossing profile of the catheter, enabling the catheter to reach and treat lesions in smaller and/or more occluded regions of an anatomical lumen. C-shaped bands can also be easier to assemble or attach to the elongate member 602, thereby reducing manufacturing costs and/or complexity of the catheter.

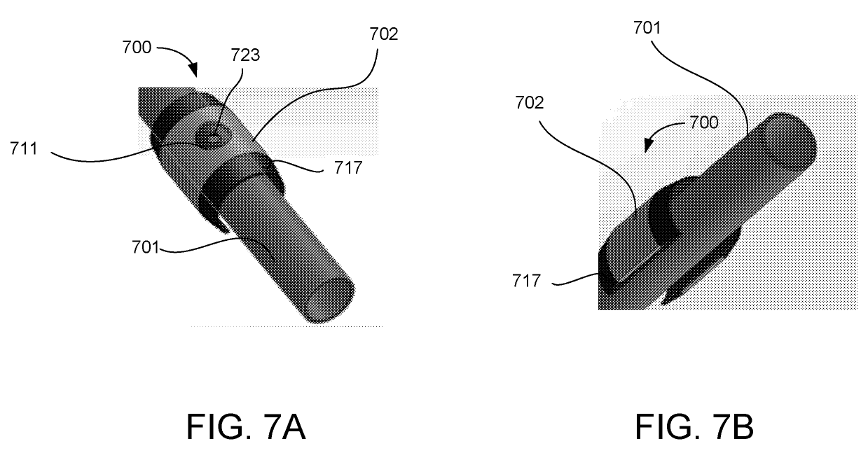

FIGS. 7A and 7B show perspective views of an example emitter 700 disposed around an elongate shaft 701, according to some implementations. In some implementations, the emitter 700 may be one example of the emitter assembly 600 of FIG. 6 or any of the emitters 310, 320, 330, 520, 530, 540, or 550 of FIGS. 3, 5A and 5B, More specifically, the emitter 700 includes an arrangement of electrodes forming spark gaps that can generate shock waves in response to voltage pulses applied thereto.

As shown in FIG. 7A, the emitter 700 includes a conductive band 702 and an insulating sheath 717 curved around an outer diameter of the elongate shaft 701. With reference to FIG. 6, the conductive band 702 may be one example of the outer electrode sheath 608, the insulating sheath 717 may be one example of the insulating sheath 606, and the elongate shaft 701 may be one example of the elongate member 602. More specifically, the conductive band 702 and the insulating sheath 717 have holes or openings 711 and 723, respectively, that are coaxially aligned with an inner electrode (not shown for simplicity) disposed between the shaft 701 and the insulating sheath 717. As described with reference to FIG. 6, the openings 711 and 723 can form a spark gap or aperture between the inner electrode and the conductive band 702 for generating shock waves. Although one spark gap is shown in the example of FIGS. 7A and 7B, in actual implementations, the emitter 700 may include one or more additional spark gaps (not shown for simplicity).

As shown in FIG. 7B, the emitter 700 does not circumscribe the elongate shaft 701. More specifically, the conductive band 702 and the insulating layer 717 for a C-shaped clamp that extends only partially around the outer diameter (or circumference) of the shaft 701. In some implementations, the emitter 700 may be crimped or clamped to the elongate shaft 701. This may reduce or eliminate bonding of the insulating layer 717 and/or the conductive band 702 to the shaft 701. Clamping or crimping the C-shaped emitter 700 can provide a stronger grip on the shaft 701 (compared to bonding) while providing a tighter (or slimmer) profile for the catheter body. The C-shaped profile of the emitter 700 also provides easier access for inserting wires along the shaft 701 and/or bond wicking, which can reduce manufacturing times.

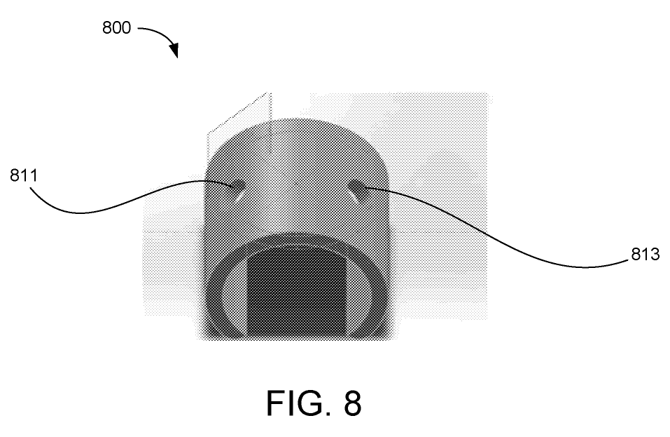

FIG. 8 shows a perspective view of an example conductive band 800 for an emitter, according to some implementations. In some implementations, the conductive band 800 may be one example of the conductive band 702 of FIGS. 7A and 7B. In the example of FIG. 8, the conductive band 800 may serve as a common electrode for multiple spark gaps.

As shown in FIG. 8, the conductive band 800 has a C-shaped profile that can curve around an outer diameter of an elongate shaft (such as the shaft 701 of FIG. 7). The conductive band 800 includes multiple openings 811 and 813 that can be coaxially aligned with respective inner electrodes (such as the inner electrode 604 of FIG. 6) to form multiple spark gaps that are radially positioned around an axis of the elongate shaft. The openings 811 and 813 can be spaced apart by any distance (or degree). In the example of FIG. 8, the openings 811 and 813 are shown to be separated by less than 180 degrees around an axis of the conductive band 800. In some other implementations, the openings 811 and 813 may be separated by up to 180 degrees around the axis of the conductive band 800. Although two openings 811 and 813 are shown in the example of FIG. 8, in actual implementations, the conductive band 800 may include one or more additional openings (not shown for simplicity).

In some implementations, an emitter may include multiple conductive bands (or curved segments) that are electrically isolated from one another. For example, each curved segment can be coupled to a different conductive channel. With reference for example to FIGS. 3 and 5A, each of the shared emitters (such as the emitters 320, 530, and 540) may include a first curved segment that forms a common electrode between spark gaps coupled to the first channel CH1 (such as the spark gaps 303 and 304 of the emitter 320, the spark gaps 503 and 504 of the emitter 530, and the spark gaps 507 and 508 of the emitter 540) and a second curved segment that forms a common electrode between spark gaps coupled to the second channel CH2 (such as the spark gaps 305 and 306 of the emitter 320, the spark gaps 505 and 506 of the emitter 530, and the spark gaps 509 and 510 of the emitter 540). Each curved segment can extend no more than 180 degrees around an outer diameter (or circumference) of the elongate shaft.

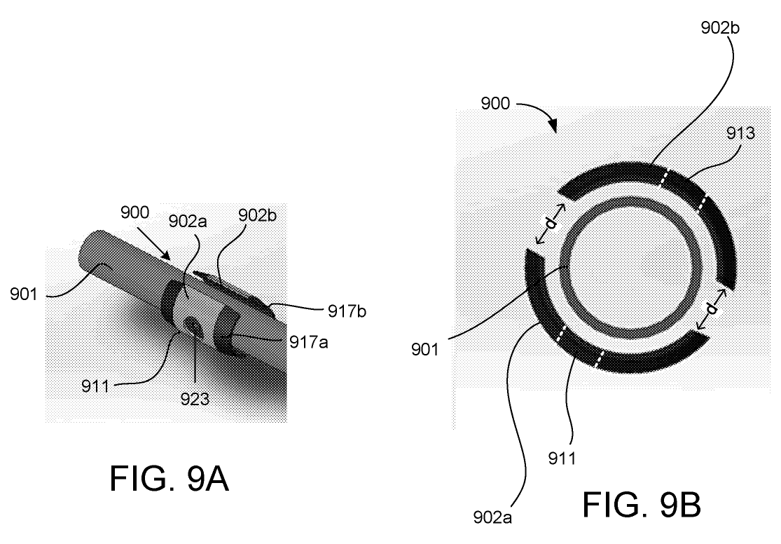

FIG. 9A shows another perspective view of an example emitter 900 disposed around an elongate shaft 901, according to some implementations. FIG. 9B shows a sectional view of the emitter depicted in FIG. 9A. In some implementations, the emitter 900 may be one example of the emitter assembly 600 of FIG. 6 or any of the emitters 310, 320, 330, 520, 530, 540, or 550 of FIGS. 3, 5A and 5B, More specifically, the emitter 900 includes an arrangement of electrodes forming spark gaps that can generate shock waves in response to voltage pulses applied thereto.

The emitter 900 includes conductive bands 902a and 902b and insulating sheaths 917a and 917b radially positioned around an outer diameter of the elongate shaft 901. With reference to FIG. 6, each of the conductive bands 902a and 902b may be one example of the outer electrode sheath 608, each of the insulating sheaths 917a and 917b may be one example of the insulating sheath 606, and the elongate shaft 901 may be one example of the elongate member 602. As shown in FIG. 9B, the edges of the conductive bands 902a and 902b are separated by a distance (d) sufficient to cause the conductive bands 902a and 902b to be electrically isolated from one another. Accordingly, the conductive band 902a and the insulating sheath 917a (not shown for simplicity in FIG. 9B) may be associated with a first emitter segment, whereas the conductive band 902b and the insulating sheath 917b (not shown for simplicity in FIG. 9B) may be associated with a second emitter segment.

The conductive band 902a and the insulating sheath 917a have holes or openings 911 and 923, respectively, that are coaxially aligned with an inner electrode (not shown for simplicity) disposed between the shaft 901 and the insulating sheath 917a. Similarly, the conductive band 902b and the insulating sheath 917b have holes or openings that are coaxially aligned with an inner electrode (not shown for simplicity) disposed between the shaft 901 and the insulating sheath 917b. As described with reference to FIG. 6, the coaxial openings between the conductive bands 902a and 902b and the insulating sheaths 917a and 917b, respectively, can form spark gaps between the inner electrodes and the conductive bands 902a and 902b for generating shock waves. Although each emitter segment is shown to include one spark gap in the example of FIGS. 9A and 9B, in actual implementations, each emitter segment may include one or more additional spark gaps (not shown for simplicity).

In some implementations, the first emitter segment (including the conductive band 902a and the inner electrode coaxially aligned with the opening 911) may be coupled to an energy source via a first conductive channel (such as the first channel CH1 of FIGS. 2–5B) and the second emitter segment (including the conductive band 902b and the inner electrode coaxially aligned with an opening 913 on the conductive band 902b) may be coupled to the energy source via a second conductive channel. In this way, the emitter 900 can be coupled to multiple conductive channels while maintaining electrical isolation between the channels.

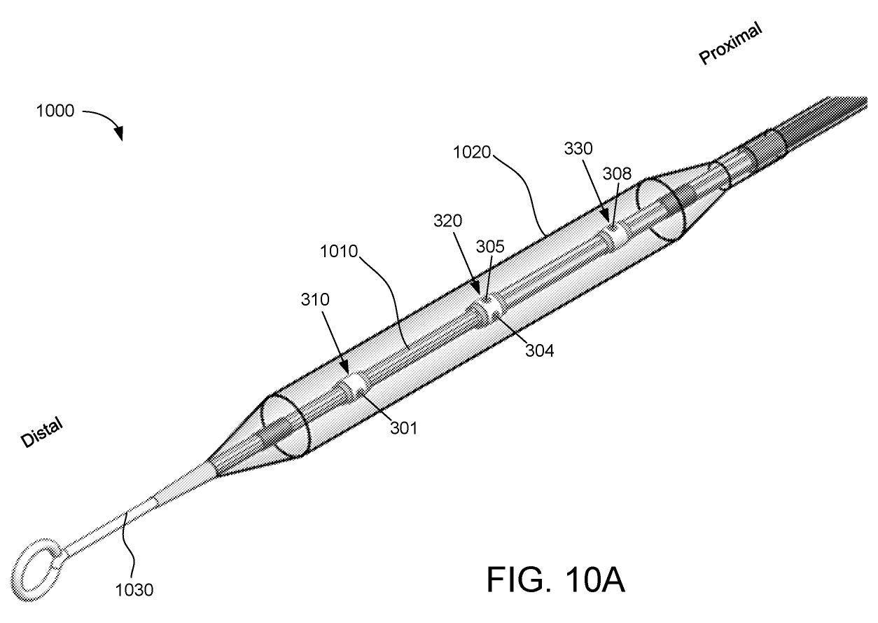

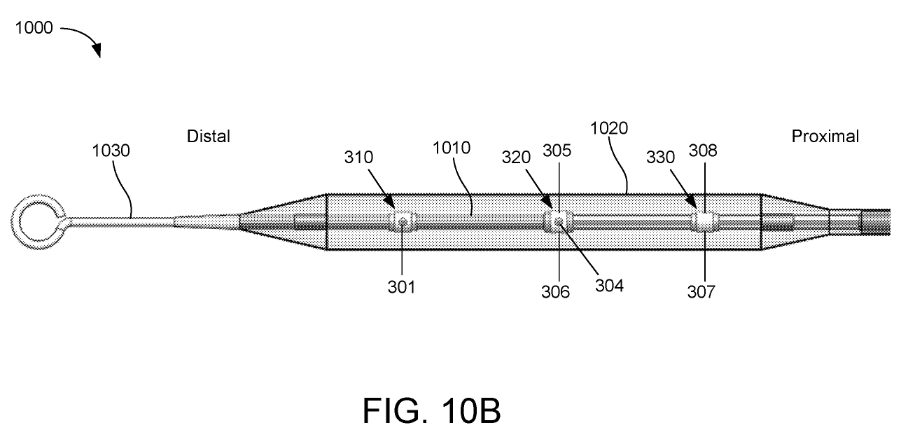

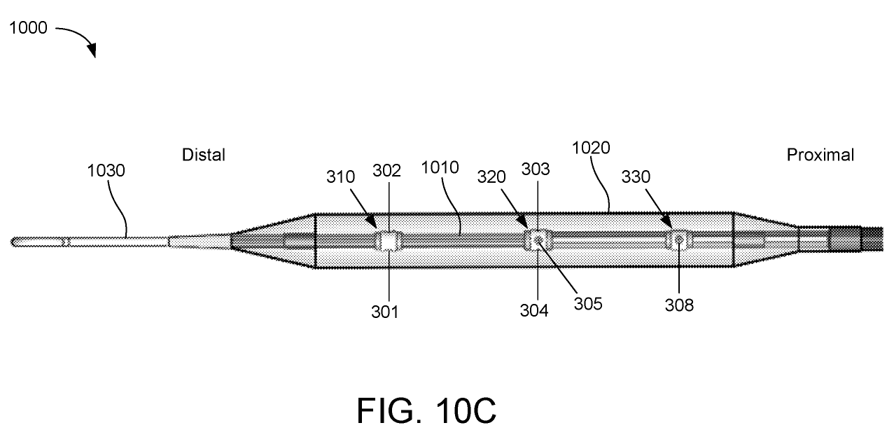

FIGS. 10A–10C show an example catheter assembly 1000, according to some implementations. More specifically, FIG. 10A shows a perspective view of the catheter assembly 1000, whereas FIGS. 10B and 10C show different side views of the catheter assembly 1000 (rotated about its axis by 90 degrees). The catheter assembly 1000 can be one example implementation of the multi-channel catheter of FIG. 3. In the example of FIGS. 10A–10C, the emitters 310, 320, and 330 are shown coupled to an elongate shaft 1010. In some implementations, the shaft 1010 may be one example of the elongate shaft 240 of FIG. 2. As described with reference to FIG. 3, the emitters 310, 320, and 330 can be electrically coupled to an energy source (such as the pulse generator 350 of FIG. 3) via multiple conductive channels CH1 and CH2.

The catheter assembly 1000 also includes an enclosure 1020 and a guidewire 1030. The enclosure 1020 is coupled to the elongate shaft 1010 such that the emitters 310, 320, and 330 are enclosed or sealed inside the enclosure 1020. In some implementations, the enclosure 1020 may be one example of the enclosure 18 of FIG. 1. For example, the enclosure 1020 can be filled with a conductive fluid that facilitates the flow of current across spark gaps in the emitters 310, 320, and 330 (such as described with reference to FIG. 1). The guidewire 1030 can be used to guide or manipulate the catheter assembly 1000 to various locations within an anatomy. In some implementations, the guidewire 1030 may be one example of the guidewire 20 of FIG. 1. For example, the guidewire 1030 can be inserted through a lumen (or inner diameter) of the shaft 1010.

In the example of FIGS. 10A–10C, the emitters 310, 320, and 330 are formed by inner and outer electrodes layered and/or positioned coaxially around the elongate shaft 1010 (such as described with reference to FIG. 6). In some implementations, each of the emitters 310, 320, and 330 may be one example of the emitter assembly 600 of FIG. 6, the emitter 700 of FIGS. 7A and 7B, or the emitter 900 of FIGS. 9A and 9B. As shown in FIGS. 10A–10C, the emitters 310, 320, and 330 are positioned at different locations along the length or axis of the elongate shaft 1010. For example, the first emitter 310 can be positioned closest to the distal end of the catheter assembly 1000, the third emitter 330 can be positioned closest to the proximal end of the catheter assembly 1000, and the second emitter 320 can be positioned in the middle of the catheter assembly 1000 (between the emitters 310 and 320).

In some implementations, the spark gaps may be positioned around the emitters to ensure optimal acoustic coverage by the shock waves. For example, as shown in FIG. 10B, the spark gaps 305 and 306 (coupled to the second channel CH2) of the second emitter 320 are separated by 180 degrees around the axis of the shaft 1010 so that the spark gaps 305 and 306 emit shock waves in opposite directions in response to voltage pulses on the second conductive channel CH2. As further shown in FIG. 10B, the spark gaps 307 and 308 of the third emitter 330 (also coupled to the second channel CH2) are also separated by 180 degrees around the axis of the shaft 1010 so that the spark gaps 307 and 308 emit shock waves in opposite directions in response to voltage pulses on the second conductive channel CH2.

As shown in FIG. 10C, the spark gaps 301 and 302 of the first emitter 310 (coupled to the first channel CH1) are separated by 180 degrees around the axis of the shaft 1010 so that the spark gaps 301 and 302 emit shock waves in opposite directions in response to voltage pulses on the first conductive channel CH1. As further shown in FIG. 10C, the spark gaps 303 and 304 (coupled to the first channel CH1) of the second emitter 320 are also separated by 180 degrees around the axis of the shaft 1010 so that the spark gaps 303 and 304 emit shock waves in opposite directions in response to voltage pulses on the first conductive channel CH1. Further, as shown in FIG. 10A, spark gaps of the second emitter 320 that are coupled to different channels (such as the spark gaps 304 and 305) are separated by 90 degrees around the axis of the shaft 1010. In this way, the arrangement of spark gaps 301–308 may reduce the frequency with which the catheter assembly 1000 is moved or repositioned to treat lesions within an anatomy.

In some implementations, the emitters 310, 320, and 330 may be rotationally aligned to enable constructive interference between the shock waves emitted by the spark gaps 301–308. For example, the spark gaps 301 and 302 of the first emitter 310 are aligned with (or facing the same direction as) the spark gaps 304 and 303, respectively, of the second emitter 320. In this way, shock waves emitted by the first spark gap 301 can constructively interfere with shock waves emitted by the fourth spark gap 304, and shock waves emitted by the second spark gap 302 can constructively interfere with shock waves emitted by the third spark gap 303, in response to voltage pulses on the first conductive channel CH1.

Similarly, the spark gaps 305 and 306 of the second emitter 320 are aligned with the spark gaps 308 and 307, respectively, of the third emitter 330. In this way, shock waves emitted by the fifth spark gap 305 can constructively interfere with shock waves emitted by the eighth spark gap 308, and shock waves emitted by the sixth spark gap 306 can constructively interfere with shock waves emitted by the seventh spark gap 307, in response to voltage pulses on the second conductive channel CH2. Such constructive interference (or combining) of shock waves can significantly increase the sonic output of the catheter assembly 1000.

In some implementations, the conductive channels CH1 and CH2 may be formed, at least in part, by wires and/or conductive traces (such as the wires 610 of FIG. 6) disposed in grooves along the elongate shaft 1010 (such as the grooves 601 of FIG. 6). The emitters 310, 320, and 330 can also be coupled to one another via wires and/or conductive traces that form part of the conductive channels CH1 or CH2. For example, the first emitter 310 can be coupled to the second emitter 320 via one or more wires of the first conductive channel CH1. Similarly, the second emitter 320 can be coupled to the third emitter 330 via one or more wires of the second conductive channel CH2. The wires can be held within the grooves or otherwise bound to the shaft 1010, for example, using heat shrink tubing (which may protect the wires while reducing the profile of the catheter assembly 1000) or any other suitable means of encapsulation.

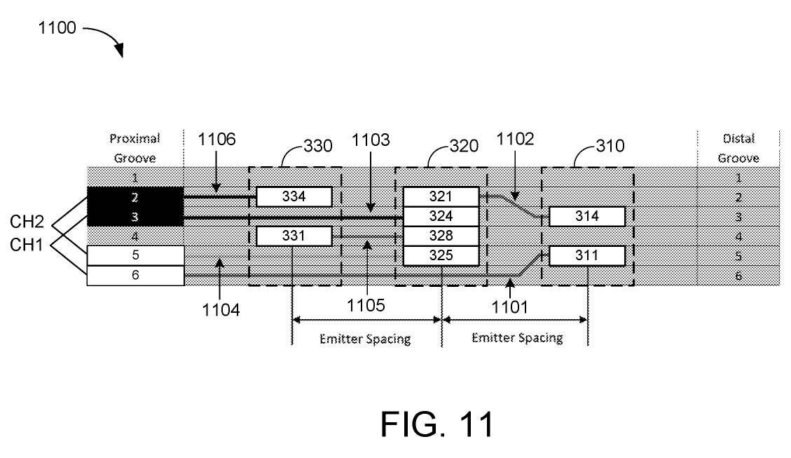

FIG. 11 shows an example wiring diagram 1100 for the catheter assembly 1000 depicted in FIGS. 10A–10C, according to some implementations. More specifically, the wiring diagram 1100 shows a number of grooves (#1–6) along the elongate shaft 1010 that can be used to carry wires and/or conductive traces. In some implementations, each of the grooves 1–6 may be one example of the groove 601 of FIG. 6. Although six grooves are shown in the example of FIG. 11, the elongate shaft 1010 can have any number of grooves.

In the example of FIG. 11, the electrodes 334 and 321 are inner electrodes disposed in groove #2, the electrodes 324 and 314 are inner electrodes disposed in groove #3, the electrodes 331 and 328 are inner electrodes disposed in groove #4, and the electrodes 325 and 311 are inner electrodes disposed in groove #5. As described with reference to FIGS. 6–9B, each of the inner electrodes 311 and 314 may form a respective spark gap with an outer electrode or conductive band of the first emitter 310, each of the inner electrodes 321, 324, 325, and 328 may form a respective spark gap with an outer electrode or conductive band of the second emitter 320, and each of the inner electrodes 331 and 334 may form a respective spark gap with an outer electrode or conductive band of the third emitter 330.

A first conductor 1101 carries current (I1) along groove #6 to the electrode 311 in groove #5. As described with reference to FIG. 3, the current I1 jumps the spark gaps 301 and 302 to reach the electrode 314 in groove #3. A second conductor 1102 carries the current I1 from the electrode 314 in groove #3 to the electrode 321 in groove #2. As described with reference to FIG. 3, the current I1 jumps the spark gaps 303 and 304 to reach the electrode 324 in groove #3. A third conductor 1103 carries the current I1 from the electrode 324 in groove #3 back to the energy source (or ground). In this way, the first conductor 1101 forms an output path of the first conductive channel CH1 (via groove #6) and the third conductor 1103 forms a return path of the first conductive channel CH1 (via groove #3).

A fourth conductor 1104 carries current (I2) along groove #5 to the electrode 325 in groove #5. As described with reference to FIG. 3, the current I2 jumps the spark gaps 305 and 306 to reach the electrode 328 in groove #4. A fifth conductor 1105 carries the current I2 from the electrode 328 in groove #4 to the electrode 331 in groove #4. As described with reference to FIG. 3, the current I2 jumps the spark gaps 307 and 308 to reach the electrode 334 in groove #2. A sixth conductor 1106 carries the current I2 from the electrode 334 in groove #2 back to the energy source (or ground). In this way, the fourth conductor 1104 forms an output path of the second conductive channel CH2 (via groove #5) and the sixth conductor 1106 forms a return path of the second conductive channel CH2 (via groove #2).

Aspects of the present disclosure recognize that the sonic output of a catheter device can be further improved or optimized by adjusting various characteristics of the emitters, including longitudinal spacing, hole-to-hole spacing, and clocking, among other examples. Longitudinal spacing refers to the separation distance between two or more emitters along the longitudinal axis of the catheter. Hole-to-hole spacing refers to the separation distance between two or more spark gaps of the same emitter. Clocking refers to the separation distance between spark gaps associated with different emitters. In the context of the catheter assembly 1000 of FIGS. 10A–10C, clocking refers to the rotational alignment between the emitters 310, 320, and 330 (such as the orientation of the spark gaps 301 and 302 of the first emitter 310 in relation to the orientation of the spark gaps 303 and 304 of the second emitter 320 and/or the orientation of the spark gaps 305 and 306 of the second emitter 320 in relation to the orientation of the spark gaps 307 and 308 of the third emitter 330).

In some implementations, the longitudinal spacing between two or more emitters of the catheter system 300 (such as between the emitters 310 and 320 and/or between the emitters 320 and 330) may be reduced to increase constructive interference between shock waves. For example, the longitudinal spacing may be ~1–4mm between the emitters 310 and 320 and/or between the emitters 320 and 330. In some other implementations, the hole-to-hole spacing of two or more spark gaps of the catheter system 300 (such as between the spark gaps 303 and 304 and/or between the spark gaps 305 and 306) may be reduced to increase constructive interference between shock waves. For example, the hole-to-hole spacing between any two spark gaps coupled to the same channel may be less than 180 degrees (or less than 90 degrees). Still further, in some implementations, the clocking between two or more emitters of the catheter system 300 (such as between the emitters 310 and 320 and/or between the emitters 320 and 330) may be aligned to increase constructive interference between shock waves. For example, each spark gap of the first emitter 310 may be positioned equidistant to both spark gaps of the second emitter 320 that are coupled to the first channel CH1. Similarly, each spark gap of the third emitter 320 may be positioned equidistant to both spark gaps of the second emitter 320 that are coupled to the second channel CH2.

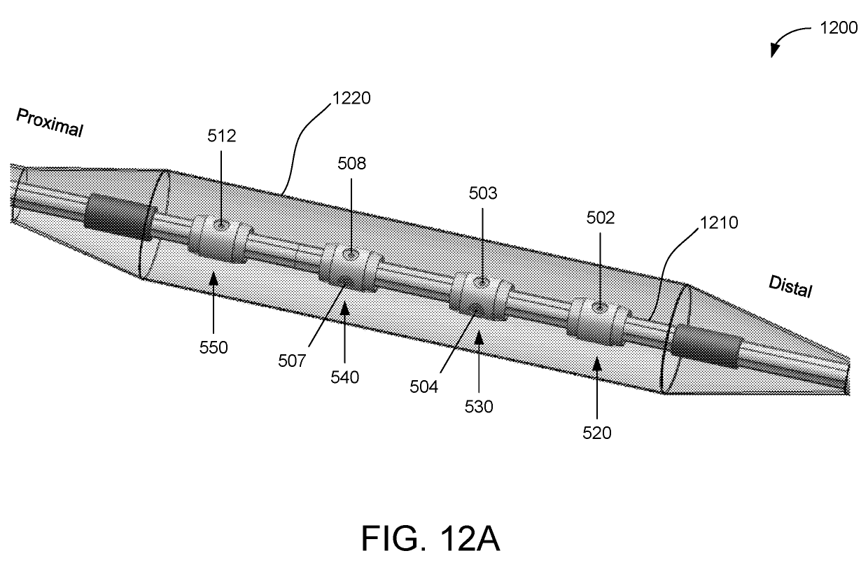

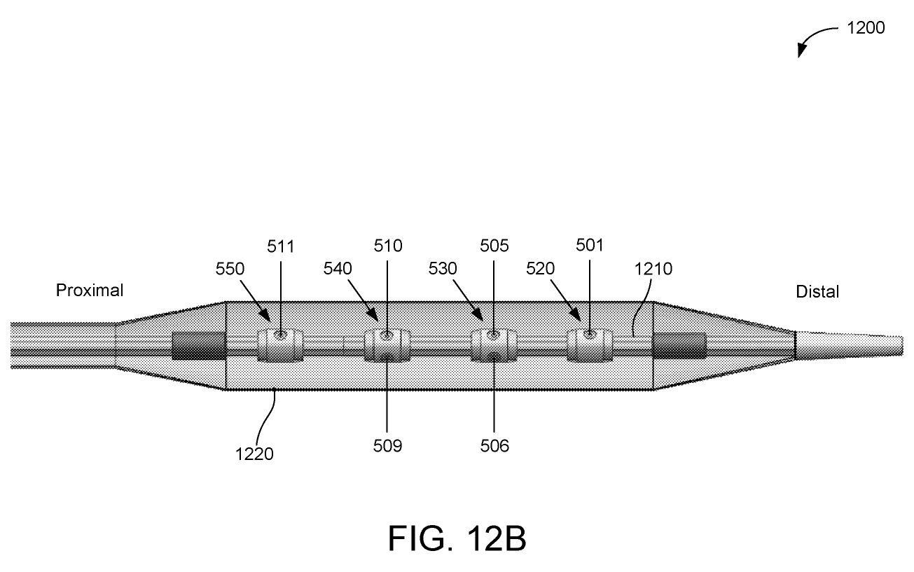

FIGS. 12A and 12B show another example catheter assembly 1200, according to some implementations. More specifically, FIG. 12A shows a perspective view of the catheter assembly 1200 and FIG. 12B shows a side view of the catheter assembly 1200. The catheter assembly 1200 is shown rotated about its axis by ~180 degrees in FIG. 12B compared to what is shown in FIG. 12A. The catheter assembly 1200 can be one example implementation of the multi-channel catheter of FIG. 5A or 5B. In the example of FIGS. 12A and 12B, the emitters 520, 530, 540, and 550 are shown coupled to an elongate shaft 1210. In some implementations, the shaft 1210 may be one example of the elongate shaft 440 of FIG. 4. As described with reference to FIGS. 5A and 5B, the emitters 520, 530, 540, and 550 can be electrically coupled to an energy source (such as the pulse generator 560) via multiple channels CH1 and CH2.

The catheter assembly 1200 also includes an enclosure 1220 coupled to the elongate shaft 1210 such that the emitters 520, 530, 540, and 550 are enclosed or sealed inside the enclosure 1220. In some implementations, the enclosure 1220 may be one example of the enclosure 18 of FIG. 1. For example, the enclosure 1220 can be filled with a conductive fluid that facilitates the flow of current across spark gaps in the emitters 520, 530, 540, and 550 (such as described with reference to FIG. 1). In some implementations, the catheter assembly 1220 also may include a guidewire (not shown for simplicity) that can be used to guide or manipulate the catheter assembly 1200 to various locations within an anatomy. For example, the guidewire can be inserted through a lumen (or inner diameter) of the shaft 1210.

In the example of FIGS. 12A and 12B, the emitters 520, 530, 540, and 550 are formed by inner and outer electrodes layered and/or positioned coaxially around the elongate shaft 1210 (such as described with reference to FIG. 6). In some implementations, each of the emitters 520, 530, 540, and 550 may be one example of the emitter assembly 600 of FIG. 6, the emitter 700 of FIGS. 7A and 7B, or the emitter 900 of FIGS. 9A and 9B. As shown in FIGS. 12A and 12B, the emitters 520, 530, 540, and 550 are positioned at different locations along the length or axis of the elongate shaft 1210. For example, the first emitter 520 can be positioned closest to the distal end of the catheter assembly 1200, the fourth emitter 550 can be positioned closest to the proximal end of the catheter assembly 1200, and the second and third emitters 530 and 540 can be positioned between the emitters 520 and 540.

In some implementations, the spark gaps may be positioned around the emitters to ensure optimal acoustic coverage by the shock waves. For example, spark gaps 501 and 502 (coupled to the first channel CH1) of the first emitter 520 are separated by 180 degrees around the axis of the shaft 1210 so that the spark gaps 501 and 502 emit shock waves in opposite directions in response to voltage pulses on the first channel CH1. Similarly, the spark gaps 511 and 512 of the fourth emitter 540 (coupled to the second channel CH2) are also separated by 180 degrees around the axis of the shaft 1210 so that the spark gaps 511 and 512 emit shock waves in opposite directions in response to voltage pulses on the second channel CH2.

On the second emitter 530, the spark gaps 503 and 505 are separated by 180 degrees around the axis of the shaft 1210 and the spark gaps 504 and 506 are also separated by 180 degrees around the axis of the shaft 1210. Further, each of the spark gaps 503 and 505 is separated from the spark gaps 504 and 506 by 90 degrees around the axis of the shaft 1210. The directionality of the shock waves emitted by the emitter 530 at any given time may depend on the coupling between the spark gaps 503–506 and the channels CH1 and CH2. For example, when the spark gaps 503 and 504 are coupled to the first channel CH1 and the spark gaps 505 and 506 are coupled to the second channel CH2 (as shown FIG. 5A), the emitter 530 may emit shock waves in orthogonal directions in response to voltage pulses on either of the channels CH1 and/or CH2. By contrast, when the spark gaps 503 and 505 are coupled to the first channel CH1 and the spark gaps 504 and 506 are coupled to the second channel CH2 (as shown in FIG. 5B), the emitter 530 may emit shock waves in opposite directions in response to voltage pulses on either of the channels CH1 and/or CH2.

On the third emitter 540, the spark gaps 508 and 510 are separated by 180 degrees around the axis of the shaft 1210 and the spark gaps 507 and 509 are also separated by 180 degrees around the axis of the shaft 1210. Further, each of the spark gaps 508 and 510 is separated from the spark gaps 507 and 509 by 90 degrees around the axis of the shaft 1210. The directionality of the shock waves emitted by the emitter 540 at any given time may depend on the coupling between the spark gaps 507–510 and the channels CH1 and CH2. For example, when the spark gaps 507 and 508 are coupled to the first channel CH1 and the spark gaps 509 and 510 are coupled to the second channel CH2 (as shown FIG. 5A), the emitter 540 may emit shock waves in orthogonal directions in response to voltage pulses on either of the channels CH1 and/or CH2. By contrast, when the spark gaps 508 and 510 are coupled to the first channel CH1 and the spark gaps 507 and 509 are coupled to the second channel CH2 (as shown in FIG. 5B), the emitter 540 may emit shock waves in opposite directions in response to voltage pulses on either of the channels CH1 and/or CH2.

In some implementations, the emitters 520, 530, 540, and 550 may be rotationally aligned to enable constructive interference between the shock waves emitted by the spark gaps 501–512. As shown in FIG. 12A, the emitters 502, 503, 508, and 512 are rotationally aligned and the emitters 504 and 507 are also rotationally aligned. As shown in FIG. 12B, the emitters 501, 505, 510, and 511 are rotationally aligned and the emitters 506 and 509 are also rotationally aligned. As described with reference to FIGS. 10A and 10B, shock waves emitted by spark gaps that are rotationally aligned can constructively interfere, increasing the sonic output of the catheter assembly 1200.

In some implementations, the conductive channels CH1 and CH2 may be formed, at least in part, by wires and/or conductive traces (such as the wires 610 of FIG. 6) disposed in grooves along the elongate shaft 1210 (such as the grooves 601 of FIG. 6). The emitters 520, 530, 540, and 550 can also be coupled to one another via wires and/or conductive traces that form part of the conductive channels CH1 or CH2. For example, the first emitter 520 can be coupled to the second and third emitter 530 and 540 via one or more wires of the first conductive channel CH1. Similarly, the fourth emitter 550 can be coupled to the second and third emitters 530 and 540 via one or more wires of the second conductive channel CH2. The wires can be held within the grooves or otherwise bound to the shaft 1210, for example, using heat shrink tubing (which may protect the wires while reducing the profile of the catheter assembly 1200) or any other suitable means of encapsulation.

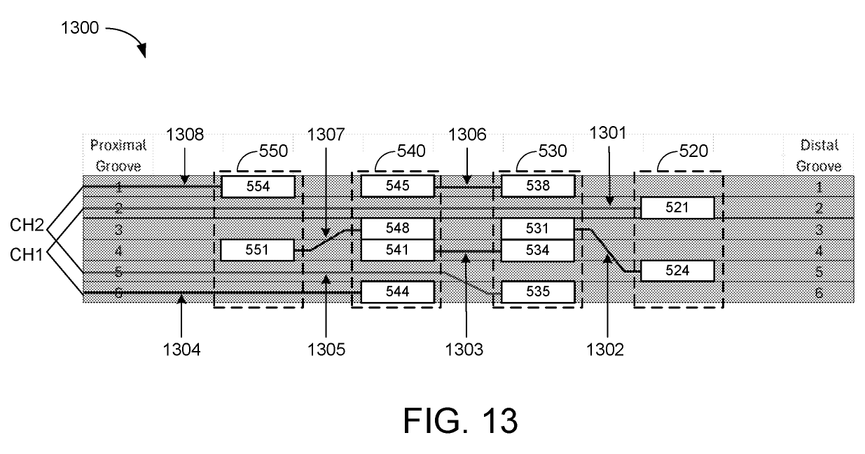

FIG. 13 shows an example wiring diagram 1300 for the catheter assembly 1200 depicted in FIGS. 12A and 12B, according to some implementations. More specifically, the wiring diagram 1300 shows a number of grooves (#1–6) along the elongate shaft 1210 that can be used to carry wires and/or conductive traces. In some implementations, each of the grooves 1–6 may be one example of the groove 601 of FIG. 6. Although six grooves are shown in the example of FIG. 13, the elongate shaft 1210 can have any number of grooves.

In the example of FIG. 13, the electrodes 551, 548, and 538 are inner electrodes disposed in groove #1, the electrode 521 is an inner electrode disposed in groove #2, the electrodes 545 and 531 are inner electrodes disposed in groove #3, the electrodes 554, 544, and 534 are inner electrodes disposed in groove #4, the electrode 524 is an inner electrode disposed in groove #5, and the electrodes 541 and 535 are inner electrodes disposed in groove #6. As described with reference to FIGS. 6–9B, each of the inner electrodes 521 and 524 may form a respective spark gap with an outer electrode or conductive band of the first emitter 520, each of the inner electrodes 538, 531, 534, and 535 may form a respective spark gap with an outer electrode or conductive band of the second emitter 530, each of the inner electrodes 548, 545, 544, and 541 may form a respective spark gap with an outer electrode or conductive band of the third emitter 540, and each of the inner electrodes 551 and 554 may form a respective spark gap with an outer electrode or conductive band of the fourth emitter 550.

A first conductor 1301 carries current (I1) along groove #2 to the electrode 521. As described with reference to FIG. 5A, the current I1 jumps the spark gaps 501 and 502 to reach the electrode 524 in groove #5. A second conductor 1302 carries the current I1 from the electrode 524 in groove #5 to the electrode 531 in groove #3. As described with reference to FIG. 5A, the current I1 jumps the spark gaps 503 and 504 to reach the electrode 534 in groove #4. A third conductor 1303 carries the current I1, along groove #4, from the electrode 534 to the electrode 541. As described with reference to FIG. 5A, the current I1 jumps the spark gaps 507 and 508 to reach the electrode 544 in groove #6. A fourth conductor 1304 carries the current I1, along groove #6, from the electrode 544 back to the energy source (or ground). In this way, the first conductor 1301 forms an output path of the first channel CH1 and the fourth conductor 1304 forms a return path of the first channel CH1.

A fifth conductor 1305 carries current (I2) along groove #5 to the electrode 535 in groove #6. As described with reference to FIG. 5A, the current I2 jumps the spark gaps 505 and 506 to reach the electrode 538 in groove #1. A sixth conductor 1306 carries the current I2, along groove #1, from the electrode 538 to the electrode 545. As described with reference to FIG. 5A, the current I2 jumps the spark gaps 509 and 510 to reach the electrode 548 in groove #3. A seventh conductor 1307 carries the current I2 from the electrode 548 in groove #3 to the electrode 551 in groove #4. As described with reference to FIG. 5A, the current I2 jumps the spark gaps 511 and 512 to reach the electrode 554 in groove #1. An eighth conductor 1308 carries the current I2, along groove #1, from the electrode 554 back to the energy source (or ground). In this way, the fifth conductor 1305 forms an output path of the second channel CH2 and the eighth conductor 1308 forms a return path of the second channel CH2.

As described with reference to FIG. 11, the sonic output of a catheter device can be further improved or optimized by adjusting various characteristics of the emitters, including longitudinal spacing, hole-to-hole spacing, and clocking, among other examples. In some implementations, the longitudinal spacing between two or more emitters of the catheter system 500 or 505 may be reduced to increase constructive interference between shock waves. In some other implementations, the hole-to-hole spacing of two or more spark gaps of the catheter system 500 or 505 may be reduced to increase constructive interference between shock waves. For example, the hole-to-hole spacing between any two spark gaps coupled to the same channel may be less than 180 degrees (or less than 90 degrees). Still further, in some implementations, the clocking between two or more emitters of the catheter system 500 or 505 may be aligned to increase constructive interference between shock waves. For example, each spark gap of the first emitter 520 may be positioned equidistant to both spark gaps of the second emitter 530 that are also coupled to the first channel CH1. Similarly, each spark gap of the fourth emitter 550 may be positioned equidistant to both spark gaps of the third emitter 540 that are also coupled to the second channel CH2.

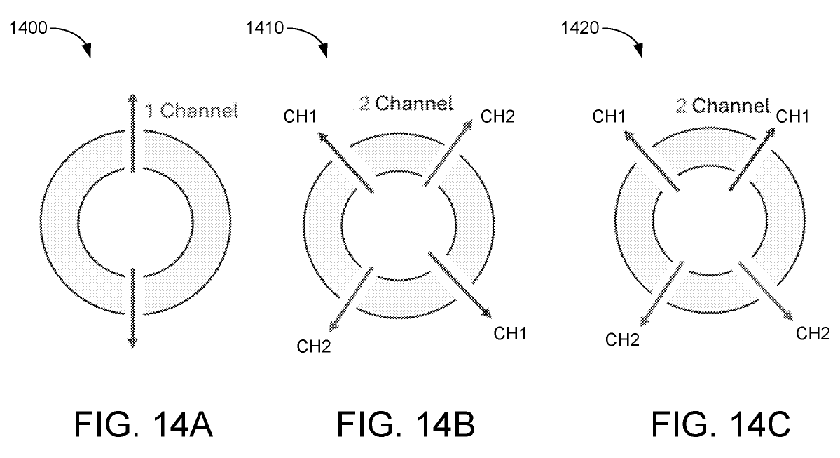

FIGS. 14A–14C show example emitter configurations, according to some implementations. More specifically, FIG. 14A shows a cross-section of an example emitter 1400 coupled to a single conductive channel, FIG. 14B shows a cross-section of an example emitter 1410 coupled to multiple conductive channels CH1 and CH2, and FIG. 14C shows another cross-section of an example emitter 1420 coupled to multiple conductive channels CH1 and CH2. In the example of FIGS. 14A–14C, the positions and orientations of spark gaps are depicted by arrows pointing radially outward from the center of each emitter.

As shown in FIG. 14A, the emitter 1400 includes a pair of spark gaps that are separated by 180 degrees about an axis of the emitter 1400 and configured to emit shock waves in opposite directions. In some implementations, the emitter 1400 may be one example of the first emitter 310 or the third emitter 330 depicted in FIGS. 10A–10C. In some other implementations, the emitter 1400 may be one example of the first emitter 520 or the fourth emitter 550 depicted in FIGS. 12A and 12B. As described with reference to FIGS. 10A–10C, 12A and 12B, this emitter configuration can provide optimal acoustic coverage for any emitter having a single pair of spark gaps.

As shown in FIG. 14B, the emitter 1410 includes two pairs of spark gaps coupled to two conductive channels CH1 and CH2, respectively, where spark gaps coupled to the same conductive channel are separated by 180 degrees about an axis of the emitter 1410. In the example of FIG. 14B, spark gaps coupled to the first conductive channel CH1 are adjacent to (and interleaved with) spark gaps coupled to the second conductive channel CH2. In some implementations, the emitter 1410 may be one example of the shared emitter 320 depicted in FIGS. 10A–10C. In some other implementations, the emitter 1410 may be one example of any of the shared emitters 530 or 540 depicted in FIGS. 12A and 12B (and described with reference to FIG. 5B). This emitter configuration can provide optimal acoustic coverage for any emitter having two pairs of spark gaps.

As shown in FIG. 14C, the emitter 1420 includes two pairs of spark gaps coupled to two conductive channel CH1 and CH2, respectively, where spark gaps coupled to the same conductive channel are separated by less than 180 degrees about an axis of the emitter 1420. In the example of FIG. 14C, spark gaps coupled to the first conductive channel CH1 are adjacent to one another and spark gaps coupled to the second conductive channel CH2 are also adjacent to one another. In some implementations, the emitter 1420 may be one example of nay of the shared emitters 530 or 540 depicted in FIGS. 12A and 12B (and described with reference to FIG. 5A). Compared to the emitter configuration of FIG. 14B, the emitter configuration of FIG. 14C reduces hole-to-hole spacing between spark gaps coupled to the same conductive channel, which can increase constructive interference between the resulting shock waves.

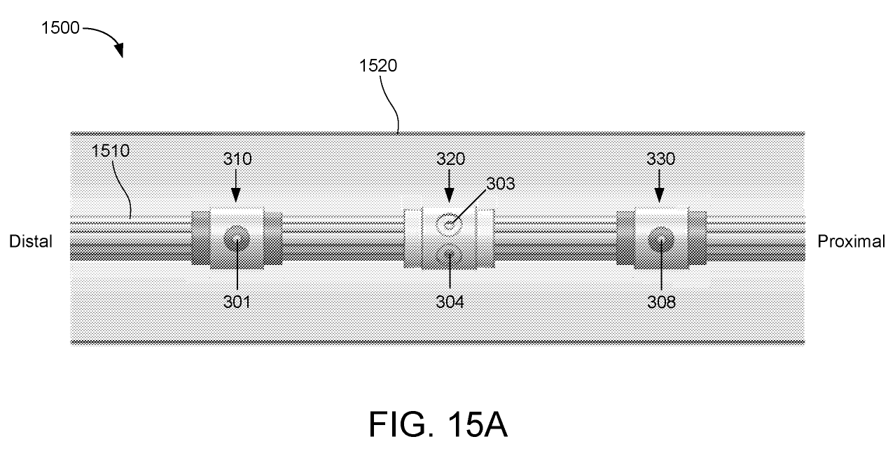

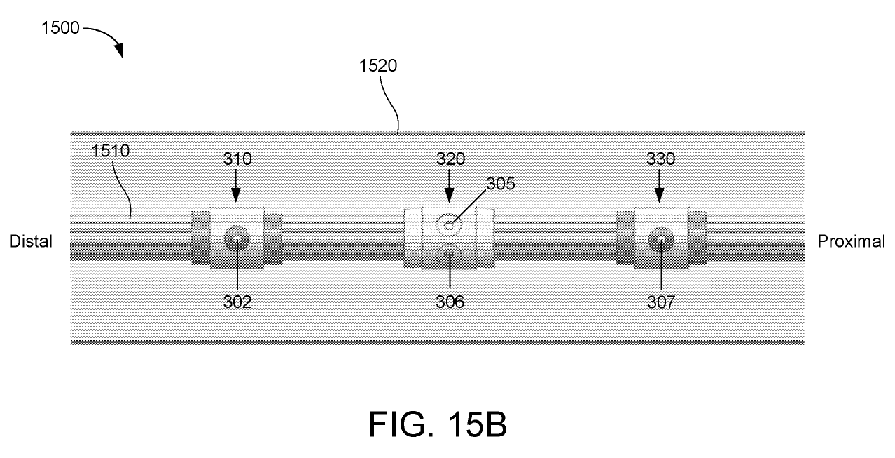

FIGS. 15A and 15B show another example catheter assembly 1500, according to some implementations. More specifically, FIGS. 15A and 15B show different side views of the catheter assembly 1500 (rotated about its axis by 180 degrees). The catheter assembly 1500 can be one example implementation of the multi-channel catheter of FIG. 3. In the example of FIGS. 15A and 15B, the emitters 310, 320, and 330 are shown coupled to an elongate shaft 1510. In some implementations, the shaft 1510 may be one example of the elongate shaft 240 of FIG. 2. As described with reference to FIG. 3, the emitters 310, 320, and 330 can be electrically coupled to an energy source (such as the pulse generator 350 of FIG. 3) via multiple conductive channels CH1 and CH2.

The catheter assembly 1500 also includes an enclosure 1520. The enclosure 1520 is coupled to the elongate shaft 1510 such that the emitters 310, 320, and 330 are enclosed or sealed inside the enclosure 1520. In some implementations, the enclosure 1520 may be one example of the enclosure 18 of FIG. 1. For example, the enclosure 1520 can be filled with a conductive fluid that facilitates the flow of current across spark gaps in the emitters 310, 320, and 330 (such as described with reference to FIG. 1). In some implementations, the catheter assembly 1500 also may include a guidewire (not shown for simplicity) that can be used to guide or manipulate the catheter assembly 1500 to various locations within an anatomy.

In the example of FIGS. 15A and 15B, the emitters 310, 320, and 330 are formed by inner and outer electrodes layered and/or positioned coaxially around the elongate shaft 1510 (such as described with reference to FIG. 6). In some implementations, each of the emitters 310, 320, and 330 may be one example of the emitter assembly 600 of FIG. 6, the emitter 700 of FIGS. 7A and 7B, or the emitter 900 of FIGS. 9A and 9B. As shown in FIGS. 15A and 15B, the emitters 310, 320, and 330 are positioned at different locations along the length or axis of the elongate shaft 1510. For example, the first emitter 310 can be positioned closest to the distal end of the catheter assembly 1500, the third emitter 330 can be positioned closest to the proximal end of the catheter assembly 1500, and the second emitter 320 can be positioned in the middle of the catheter assembly 1500 (between the emitters 310 and 320). Compared to FIGS. 10A–10C, the longitudinal spacing of emitters 310, 320, and 330 is significantly reduced in the example of FIGS. 15A and 15B, which can increase constructive interference between shock waves emitted by the catheter assembly 1500.

In some implementations, the spark gaps may be positioned around the emitters to ensure optimal acoustic coverage by the shock waves. For example, the spark gaps 301 and 302 of the first emitter 310 (coupled to the first channel CH1) can be separated by 180 degrees around the axis of the shaft 1510 so that the spark gaps 301 and 302 emit shock waves in opposite directions responsive to voltage pulses on the first conductive channel CH1. Similarly, the spark gaps 307 and 308 of the third emitter 330 (coupled to the second channel CH2) can also be separated by 180 degrees around the axis of the shaft 1510 so that the spark gaps 307 and 308 emit shock waves in opposite directions responsive to voltage pulses on the second conductive channel CH2.

In some implementations, the hole-to-hole spacing between spark gaps of the shared emitter 320 may be configured to increase sonic output (compared to the shared emitter 320 of FIGS. 10A–10C). For example, as shown in FIG. 15A, the spark gaps 303 and 304 (coupled to the first channel CH1) of the second emitter 320 are separated by less than 180 degrees (and/or less than 90 degrees) around the axis of the shaft 1510 so that the shock waves emitted by the spark gaps 303 and 304 can constructively interfere with one another in response to voltage pulses on the first conductive channel CH1. Similarly, as shown in FIG. 15B, the spark gaps 305 and 306 (coupled to the second channel CH2) of the second emitter 320 are also separated by less than 180 degrees (and/or less than 90 degrees) around the axis of the shaft 1510 so that the shock waves emitted by the spark gaps 305 and 306 can constructively interfere with one another in response to voltage pulses on the second conductive channel CH2. In some implementations, shock waves generated by the emitter 320 may have a sonic pressure significantly greater than 4.2 Megapascals (MPa) at a distance of ~3mm away from the source.

In some implementations, the emitters 310, 320, and 330 may be rotationally aligned (such as via clocking) to further improve constructive interference between the shock waves emitted by the spark gaps 301–308. For example, as shown in FIG. 15A, the spark gap 301 of the first emitter 310 is substantially aligned with the spark gaps 303 and 304 of the second emitter 320 (such that the first spark gap 301 is oriented in a direction between the orientation of the third spark gap 303 and the orientation of the fourth spark gap 304). In this way, shock waves emitted by the first spark gap 301 can constructively interfere with shock waves emitted by the third spark gap 303, and with shock waves emitted by the fourth spark gap 304, in response to voltage pulses on the first conductive channel CH1.

Similarly, as shown in FIG. 15B, the spark gap 307 of the third emitter 330 is substantially aligned with the spark gaps 305 and 306 of the second emitter 320 (such that the seventh spark gap 307 is oriented in a direction between the orientation of the fifth spark gap 305 and the orientation of the sixth spark gap 306). In this way, shock waves emitted by the seventh spark gap 307 can constructively interfere with shock waves emitted by the fifth spark gap 305, and with shock waves emitted by the sixth spark gap 306, in response to voltage pulses on the second conductive channel CH2. Compared to FIGS. 10A–10C, the hole-to-hole spacing and clocking of emitters 310, 320, and 330 in the example of FIGS. 15A and 15B can significantly increase constructive interference between shock waves emitted by the catheter assembly 1500.

In some implementations, the conductive channels CH1 and CH2 may be formed, at least in part, by wires and/or conductive traces (such as the wires 610 of FIG. 6) disposed in grooves along the elongate shaft 1510 (such as the grooves 601 of FIG. 6). The emitters 310, 320, and 330 can also be coupled to one another via wires and/or conductive traces that form part of the conductive channels CH1 or CH2. For example, the first emitter 310 can be coupled to the second emitter 320 via one or more wires of the first conductive channel CH1. Similarly, the second emitter 320 can be coupled to the third emitter 330 via one or more wires of the second conductive channel CH2. The wires can be held within the grooves or otherwise bound to the shaft 1510, for example, using heat shrink tubing (which may protect the wires while reducing the profile of the catheter assembly 1500) or any other suitable means of encapsulation.

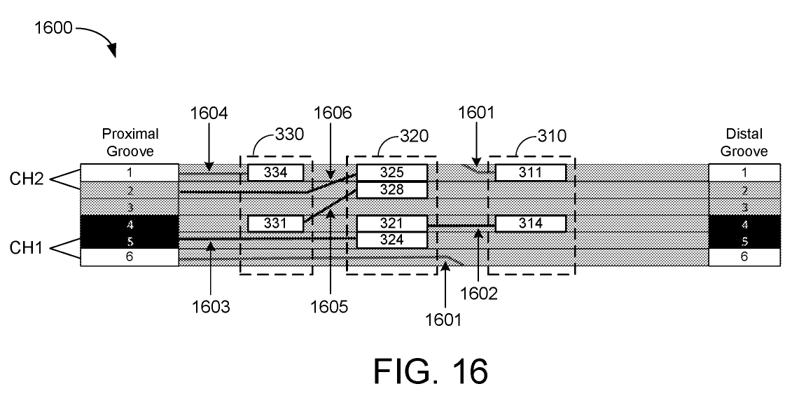

FIG. 16 shows an example wiring diagram 1600 for the catheter assembly 1500 depicted in FIGS. 15A and 15B, according to some implementations. More specifically, the wiring diagram 1600 shows a number of grooves (#1–6) along the elongate shaft 1510 that can be used to carry wires and/or conductive traces. In some implementations, each of the grooves may be one example of the groove 601 of FIG. 6. Although six grooves are shown in the example of FIG. 16, the elongate shaft 1510 may have fewer or more grooves in some other implementations.

In the example of FIG. 16, the electrodes 334, 325, and 311 are inner electrodes disposed in groove #1, the electrodes 328 is an inner electrode disposed in groove #2, the electrodes 331, 321, and 314 are inner electrodes disposed in groove #4, and the electrode 324 is an inner electrode disposed in groove #5. As described with reference to FIGS. 6–9B, 15A and 15B, each of the inner electrodes 311 and 314 may form a respective spark gap with an outer electrode or conductive band of the first emitter 310, each of the inner electrodes 321, 324, 325, and 328 may form a respective spark gap with an outer electrode or conductive band of the second emitter 320, and each of the inner electrodes 331 and 334 may form a respective spark gap with an outer electrode or conductive band of the third emitter 330.

A first conductor 1601 carries current (I1) along groove #6 to the electrode 311 in groove #1. As described with reference to FIG. 3, the current I1 jumps the spark gaps 301 and 302 to reach the electrode 314 in groove #4. A second conductor 1602 carries the current I1 from the electrode 314 in groove #4 to the electrode 321 in groove #4. As described with reference to FIG. 3, the current I1 jumps the spark gaps 303 and 304 to reach the electrode 324 in groove #5. A third conductor 1603 carries the current I1 from the electrode 324 in groove #5 back to the energy source (or ground). In this way, the first conductor 1601 forms an output path of the first conductive channel CH1 (via groove #6) and the third conductor 1603 forms a return path of the first conductive channel CH1 (via groove #5).

A fourth conductor 1604 carries current (I2) along groove #1 to the electrode 334 in groove #1. In the example of FIG. 16, the current I2 flows in the opposite direction of the arrow shown in FIG. 3. Accordingly, the current I2 jumps the spark gaps 308 and 307 to reach the electrode 331 in groove #4. A fifth conductor 1605 carries the current I2 from the electrode 331 in groove #4 to the electrode 328 in groove #2. With reference for example to FIG. 3, the current I2 jumps the spark gaps 306 and 305 to reach the electrode 325 in groove #1. A sixth conductor 1606 carries the current I2 from the electrode 325 in groove #1 back to the energy source (or ground). In this way, the fourth conductor 1604 forms an output path of the second conductive channel CH2 (via groove #1) and the sixth conductor 1606 forms a return path of the second conductive channel CH2 (via groove #2).

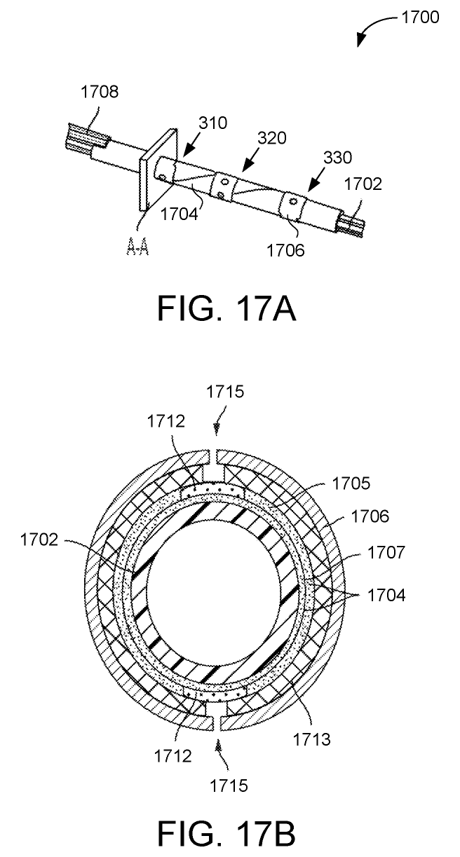

FIGS. 17A and 17B show another example catheter assembly 1700, according to some implementations. More specifically, FIG. 17A shows a perspective view of the catheter assembly 1700 and FIG. 17B shows a sectional view of the catheter assembly (along the plane A_A). The catheter assembly 1700 can be one example implementation of the multi-channel catheter of FIG. 3. In the example of FIGS. 17A and 17B, the emitters 310, 320, and 330 are shown coupled to an elongate shaft 1702. In some implementations, the shaft 1702 may be one example of the elongate shaft 240 of FIG. 2. As described with reference to FIG. 3, the emitters 310, 320, and 330 can be electrically coupled to an energy source (such as the pulse generator 350 of FIG. 3) via multiple conductive channels CH1 and CH2.

In the example of FIGS. 17A and 17B, the catheter assembly 1700 includes a flex circuit 1704 having one or more metallic traces (not shown in FIG. 17A) that form the inner electrodes for each of the spark gaps of the emitters 310, 320, and 330. More generally, the electrodes can be any electrically conducting element (such as conductive traces, wires, and/or other suitable metallic objects or surfaces). The catheter assembly 1700 includes one or more conductive bands 1706 that form one or more outer electrodes of one or more electrode pairs. In some implementations, the flex circuit 1704 may be helically wrapped around the elongate shaft 1702. The flex circuit 1704 can have any suitable number of turns, depending on the length of the catheter assembly 1700, the number of spark gaps on each of the emitters 310, 320, and 330, and/or the arrangement of the spark gaps. In some other implementations, the flex circuit 1704 may be wrapped around the elongate shaft 1702 (such as in an overlapping configuration) with successive electrically joined flex circuits, or with a staircase-shaped or rectangularized flex circuit.

The catheter assembly 1700 further includes one or more wires 1708 coupled to the flex circuit 1704, for example, to supply voltage or current to the emitters 310, 320, and 330. The wires 1708 can be coupled to the flex circuit 1704 such that the wires 1708 provide voltage or current to the metal traces of the metallization layer. In some implementations, the catheter assembly 1700 may include a respective pair of wires for each conductive channel CH1 and CH2. As shown in FIGS. 17A and 17B, the conductive bands 1706 are mounted around the flex circuit 1704. In some implementations, each of the conductive bands 1706 may be one example of the outer electrode sheath 608 of FIG. 6, the conductive band 702 of FIGS. 7A and 7B, the conductive band 800 of FIG. 8, or any of the conductive bands 902a or 902b of FIGS. 9A and 9B.

FIG. 17B shows traces 1712 of a metallization layer 1705 formed on a flexible base layer 1707 of the flex circuit 1704 and an insulation layer 1713 of the flex circuit 1704 that overlays the metallization layer 1705. The flex circuit 1704 is wrapped around the elongate shaft 1702 such that two traces 1712 of the metallization layer of the flex circuit 1704 are disposed at opposite locations. This illustration is exemplary, as the traces 1712 can be positioned and oriented in any desired location and/or orientation. The conductive band 1706 is separated from the metallization layer 1705 by the insulation layer 1713.

The traces 1712 and conductive band 1706 form two electrode pairs 1715, each formed form a respective trace 1712 (forming an inner electrode) and apportion of the conductive band 1706 (forming an outer electrode). This is provided for example only, as an emitter may include any number of electrode pairs (or spark gaps). In the example of FIG. 17B, the electrode pairs 1715 are separated by 180 degrees about an axis of the elongate shaft 1702. In some other implementations, the electrode pairs 1715 may be separated by less than 180 (such as described with reference to FIGS. 15A and 15B). Further, the catheter assembly 1700 may include any number of traces 1712 suitable for the size and/or function of the catheter assembly 1700. The traces can be formed from an conductive material (such as metal).

In some implementations, the traces 1712 of the flex circuit 1704 may be formed via a deposition metallization process. For example, the traces 1712 may be formed by vacuum metallizing, wherein a metallic material is heated to its boiling point in a vacuum chamber and then deposited via condensation on a substrate. The traces 1712 also may be formed via a thermal spray process, wherein metallic material is heated and then sprayed onto a substrate. The traces also may be formed via a cold spray process, wherein cold sprayable metallic material is applied to a substrate. When cold spraying, the metallic material may comprise a composite of metallic powder, a water-based binder, and a hardening agent. The traces 1712 also may be formed via a hot-dip process, wherein a substrate is dipped into a metallic bath comprising molten metallic material and then removed and allowed to harden.

The insulation layer 1713 may be formed from a non-conductive insulating material that prevents unintended current flow between certain regions of the flex circuit 1704 (such as portions of the metallic traces 1712) and the conductive band 1706. For example, the insulation layer 1713 can prevent current from flowing between the trace 1712 and the adjacent region of the conductive band 1706 of each electrode pair 1715. In some implementations, the insulation layer 1713 may be formed from silicon nitride (SiNₓ), silicon oxide (SiOₓ), polyimide, polytetrafluorethylene (PTFE), diamond-like carbon (DLC), thermoplastic polyurethane (TPU), or any combination thereof.

In some implementations, the flex circuit 1704 may include a flexible substrate, such as polyimide, polyether ether ketone (PEEK), or a transparent conductive polyester film, with the traces 1712 formed on the surface of the flexible substrate. In such implementations, the flex circuit 1704 can be a single-sided flex circuit (such as a flex circuit having a single conductive layer), a double-sided flex circuit (such as a flex circuit having two conductive layers), or a multilayer flex circuit (such as a flex circuit having three or more conductive layers).

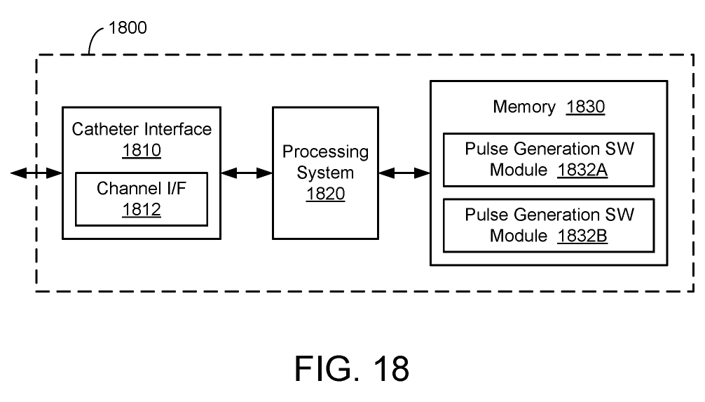

FIG. 18 shows a block diagram of an example pulse generator 1800 for a medical system, according to some implementations. In some implementations, the pulse generator 1800 may be one example of any of the energy sources 28, 250, or 450 of FIGS. 1, 2, and 4 or the pulse generators 350 or 560 of FIGS. 3, 5A, and 5B, More specifically, the pulse generator 1800 is configured to deliver energy pulses to a medical instrument (such as a lithotripsy catheter) via multiple channels.

The pulse generator 1800 includes a catheter interface 1810, a processing system 1820, and a memory 1830. The catheter interface 1810 is configured to communicate with one or more components of the catheter. More specifically, the catheter interface 1810 includes a channel interface (I/F) 1812 for communicating with subsets of emitters and/or electrodes coupled to each of the channels. The memory 1830 includes a non-transitory computer-readable medium (including one or more nonvolatile memory elements, such as EPROM, EEPROM, Flash memory, or a hard drive, among other examples) that can store various software (SW) modules.

In some implementations, the memory 1830 may store the following software modules: a first pulse generation SW module 1832A to output one or more first energy pulses on a first channel coupled to a first emitter and a second emitter of a medical instrument, where each of the first and second emitters is configured to produce acoustic waves responsive to the one or more first energy pulses; and a second pulse generation SW module 1832B to output one or more second energy pulses on a second channel coupled to the second emitter and a third emitter of the medical instrument, where each of the second and third emitters is configured to produce acoustic waves responsive to the one or more second energy pulses. In some implementations, the first and second pulse generation SW modules 1832A and 1832B may be combined into a single SW module.

The processing system 1820 may include any suitable one or more processors capable of executing scripts or instructions of one or more software programs stored in the pulse generator 1800 (such as in the memory 1830). For example, the processing system 1820 can execute the first pulse generation SW module 1832A to output one or more first energy pulses on a first channel coupled to a first emitter and a second emitter of a medical instrument, where each of the first and second emitters is configured to produce acoustic waves responsive to the one or more first energy pulses. The processing system 1820 can also execute the second pulse generation SW module 1832B to output one or more second energy pulses on a second channel coupled to the second emitter and a third emitter of the medical instrument, where each of the second and third emitters is configured to produce acoustic waves responsive to the one or more second energy pulses.

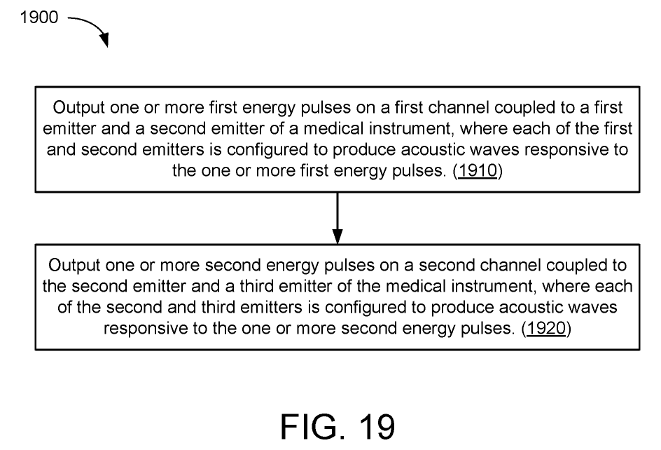

FIG. 19 shows an illustrative flowchart depicting an example operation 1900 that can be performed by a pulse generator for a medical system, according to some implementations. In some implementations, the pulse generator may be one example of pulse generator 1800 of FIG. 18. More specifically, the pulse generator is configured to deliver energy pulses to a medical instrument (such as a lithotripsy catheter) via multiple channels.

The pulse generator outputs one or more first energy pulses on a first channel coupled to a first emitter and a second emitter of a medical instrument, where each of the first and second emitters is configured to produce acoustic waves responsive to the one or more first energy pulses (1910). The pulse generator also outputs one or more second energy pulses on a second channel coupled to the second emitter and a third emitter of the medical instrument, where each of the second and third emitters is configured to produce acoustic waves responsive to the one or more second energy pulses (1920). In some implementations, the outputting of the one or more first energy pulses may be interleaved with the outputting of the one or more second energy pulses.

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described herein. Whether such functionality is implemented in hardware or software depends upon the particular application and design constraints imposed on the overall system.

In the foregoing specification, implementations have been described with reference to specific examples thereof. It will, however, be evident that various modifications and changes may be made thereto without departing from the broader scope of the disclosure as set forth in the appended claims. The specification and drawings are, accordingly, to be regarded in an illustrative sense rather than a restrictive sense.

As used herein, a phrase referring to “at least one of” a list of items refers to any combination of those items, including single members. As an example, “at least one of: a, b, or c” is intended to cover: a, b, c, a-b, a-c, b-c, and a-b-c.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

What is claimed is:

1. A medical instrument, comprising:
an elongate shaft having a plurality of transmission media disposed thereon forming a first channel and a second channel;
a first emitter disposed on the elongate shaft and configured to produce acoustic waves responsive to energy pulses on the first channel;
a second emitter disposed on the elongate shaft and configured to produce acoustic waves responsive to energy pulses on the second channel; and
a third emitter disposed on the elongate shaft and configured to produce acoustic waves responsive to the energy pulses on each of the first channel and the second channel.

2. The medical instrument of claim 1, wherein the first emitter, the second emitter, and the third emitter are disposed at different locations along a length of the elongate shaft.

3. The medical instrument of claim 2, wherein the third emitter is located between the first emitter and the second emitter.

4. The medical instrument of claim 1, wherein the first emitter comprises:
a first spark gap positioned radially about an axis of the elongate shaft and configured to emit acoustic waves responsive to the energy pulses on the first channel; and
a second spark gap radially offset from the first spark gap relative to the axis of the elongate shaft and configured to emit acoustic waves responsive to the energy pulses on the first channel.

5. The medical instrument of claim 4, wherein the first spark gap and the second spark gap share a common electrode comprising a conductive band that extends at least partially around an outer diameter of the elongate shaft.

6. The medical instrument of claim 4, wherein the third emitter comprises:
a third spark gap positioned radially about the axis of the elongate shaft and configured to emit acoustic waves responsive to the energy pulses on the first channel;
a fourth spark gap radially offset from the third spark gap relative to the axis of the elongate shaft and configured to emit acoustic waves responsive to the energy pulses on the first channel;
a fifth spark gap radially offset from the third spark gap and the fourth spark gap relative to the axis of the elongate shaft and configured to emit acoustic waves responsive to the energy pulses on the second channel; and
a sixth spark gap radially offset from the third spark gap, the fourth spark gap, and the fifth spark gap relative to the axis of the elongate shaft and configured to emit acoustic waves responsive to the energy pulses on the second channel.

7. The medical instrument of claim 6, wherein the third, fourth, fifth, and sixth spark gaps share a common electrode.

8. The medical instrument of claim 7, wherein the common electrode comprises a cylindrical band that circumscribes an outer diameter of the elongate shaft.

9. The medical instrument of claim 6, wherein the third spark gap and the fourth spark gap share a first common electrode, and the fifth spark gap and the sixth spark gap share a second common electrode that is electrically isolated from the first common electrode.

10. The medical instrument of claim 9, wherein each of the first common electrode and the second common electrode comprises a curved band that extends partially around an outer diameter of the elongate shaft.

11. The medical instrument of claim 6, wherein the radial offset between the third spark gap and the fourth is equal to180 degrees and the radial offset between the fifth spark gap and the sixth spark gap is equal to180 degrees.

12. The medical instrument of claim 6, wherein the radial offset between the third spark gap and the fourth spark gap is equal to180 degrees and the radial offset between the fifth spark gap and the sixth spark gap is equal to180 degrees.

13. The medical instrument of claim 12, wherein the first spark gap is oriented in a direction between an orientation of the third spark gap and an orientation of the fourth spark gap, and the second spark gap is oriented in a direction between an orientation of the fifth spark gap and an orientation of the sixth spark gap.

14. The medical instrument of claim 6, wherein the second emitter comprises:
a seventh spark gap positioned radially about an axis of the elongate shaft and configured to emit acoustic waves responsive to the energy pulses on the second channel; and
an eighth spark gap radially offset from the seventh spark gap relative to the axis of the elongate shaft and configured to emit acoustic waves responsive to the energy pulses on the second conductive channel.

15. The medical instrument of claim 14, wherein the seventh spark gap is oriented in the same direction as the first spark gap and the eighth spark gap is oriented in the same direction as the second spark gap.

16. The medical instrument of claim 14, wherein the seventh spark gap and the eighth spark gap are oriented in directions orthogonal to an orientation of the first spark gap and an orientation of the second spark gap.

17. The medical instrument of claim 14, further comprising:
a fourth emitter configured to produce acoustic waves responsive to the energy pulses on each of the first conductive channel and the second conductive channel.

18. The medical instrument of claim 17, wherein the fourth emitter comprises:
a ninth spark gap positioned radially about the axis of the elongate shaft and configured to emit acoustic waves responsive to the energy pulses on the first channel;
a tenth spark gap radially offset from the ninth spark gap relative to the axis of the elongate shaft and configured to emit acoustic waves responsive to the energy pulses on the first channel;
an eleventh spark gap radially offset from the ninth spark gap and the tenth spark gap relative to the axis of the elongate shaft and configured to emit acoustic waves responsive to the energy pulses on the second channel; and
a twelfth spark gap radially offset from the ninth spark gap, the tenth spark gap, and the eleventh spark gap relative to the axis of the elongate shaft and configured to emit acoustic waves responsive to the energy pulses on the second channel.

19. A method performed by a pulse generator for a medical system, comprising:
outputting one or more first energy pulses on a first channel coupled to a first emitter and a second emitter of a medical instrument, each of the first and second emitters configured to produce acoustic waves responsive to the one or more first energy pulses; and
outputting one or more second energy pulses on a second channel coupled to the second emitter and a third emitter of the medical instrument, each of the second and third emitters configured to produce acoustic waves responsive to the one or more second energy pulses.

20. The system of claim 19, wherein the outputting of the one or more first energy pulses are interleaved with the outputting of the one or more second energy pulses.
